SEQUENCE LISTING (6 sequences)

SEQ ID NO: 1 - protein, 259 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 2 - protein, 229 aa, Homo sapiens
APCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 3 - protein, 258 aa, Homo sapiens
SEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSKALHVTNIKKWKEPCRIELYRVVESLAKAQETSGEEISKFYLPNCNKNGFYHSRQCETSMDGEAGLCWCVYPWNGKRIPGSPEIRGDPNCQIYFNVQN

SEQ ID NO: 4 - protein, 137 aa, Homo sapiens
APWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSK

SEQ ID NO: 5 - protein, 162 aa, Homo sapiens
MSEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSK

SEQ ID NO: 6 - protein, 161 aa, Homo sapiens
SEVPVARVWLVLLLLTVQVGVTAGAPWQCAPCSAEKLALCPPVSASCSEVTRSAGCGCCPMCALPLGAACGVATARCARGLSCRALPGEQQPLHALTRGQGACVQESDASAPHAAEAGSPESPESTEITEEELLDNFHLMAPSEEDHSILWDAISTYDGSK

(19) United States
(12) Patent Application Publication
(10) Pub. No.: US 2022/0057407 A1
(43) Pub. Date: Feb. 24, 2022

(54) METHOD FOR DETECTING IMMINENT CHILDBIRTH

(71) Applicant: BIOSYNEX, Illkirch-Graffenstaden (FR)

(72) Inventors: Thierry PAPER, Strasbourg (FR); Capucine BLIN, Wolfisheim (FR)

(21) Appl. No.: 17/488,906

(22) Filed: Sep. 29, 2021

Related U.S. Application Data
(63) Continuation of application No. 16/080,189, filed on Aug. 27, 2018, now abandoned.

(30) Foreign Application Priority Data
Feb. 26, 2016 (FR) 1651626

(51) Int. Cl. G01N 33/68; G01N 33/74

(52) U.S. Cl. CPC G01N 33/6869; G01N 33/74; G01N 33/689; G01N 2800/60; G01N 2333/4745; G01N 2333/5412; G01N 2800/368

(57) ABSTRACT

Some embodiments are directed to a method for the in vitro detection/prediction of imminent childbirth based on the detection of at least two of the following markers: insulin-like growth factor-binding protein 1 (IGFBP-1), a N-terminal fragment of insulin-like growth factor-binding protein 1, and interleukin 6 (IL-6). Some embodiments are also directed to a device for the implementations of the method. Some embodiments are applicable in the medical field, in the diagnostic field, and in particular in the obstetric field.

### CROSS REFERENCE TO RELATED APPLICATIONS

This application is a Continuation of U.S. patent application Ser. No. 16/080,189, filed on Aug. 27, 2018, which is a U.S. National Stage Entry of PCT International Patent Application No. PCT/FR2017/050,002, filed on Jan. 3, 2017, which claims priority to French Patent Application No. 1651626, filed Feb. 26, 2016. The content of each of the prior applications are hereby incorporated in their entireties by reference.

### BACKGROUND

Some embodiments relate to a method for the in vitro detection/prediction of imminent childbirth and to a device the implementation of the method.

Some embodiments are applicable in the medical field, in the diagnostic field, and in particular in the obstetric field.

In the description below, the references between refer to the list of references presented at the end of the text.

During pregnancy, women can be subjected to various factors and/or events capable of endangering the pregnancy and/or of causing a preterm birth or of triggering childbirth.

Preterm birth is a risk factor both for the mother and also for the child to be born.

Preterm birth is in 50% of cases preceded by a threat of preterm birth, that is to say 2 the occurrence of a combination of cervical modifications and of regular and painful uterine contractions between the 22nd and 36th weeks of amenorrhea. Preterm births are responsible for 75% of neonatal mortality, half of neurological defects and represent the primary cause of hospitalization during pregnancy, that is to say approximately 60 000 hospitalizations per year.

Depending on the date of childbirth and on the ac of the fetus, there are various types of maternity ward in order to provide newborns with the corresponding care.

Level I maternity wards have an obstetric unit and enable the treatment of normal pregnancies. These maternity wards provide a pediatric presence which allows examination of the newborn and the treatment, for the mother, of a certain number of frequent and non-serious situations.

Level II maternity wards have an obstetric unit and a neonatology unit. They allow the treatment of moderate-risk pregnancies and of newborns requiring particular monitoring, but not intensive care.

Level III maternity wards have an obstetric unit, a neonatology unit and a neonatal intensive care unit. They enable the treatment of high-risk pregnancies and newborns presenting a risk at birth; they may in particular be extremely preteen births at under 33 weeks.

A preterm birth can occur in women the to be at risk, for example women with hypertension or gestational diabetes, but also in women who do not have such factors, termed low-risk women.

Numerous studies and research projects have been carried out on preterm labor and/or birth. However, there is currently no totally reliable method for detecting this preterm labor/birth. The known for the treatment of preterm labor/birth are pharmacological which are used once labor has commenced. These involve, for example, in the case of preterm labor at 30 weeks amenorrhea, a pharmacological protocol with administration of a tocolytic, for example tractocile for 48 hours, of nifedipine, accompanied where appropriate by an administration of corticosteroids for maturation of the lungs of the fetus.

### SUMMARY

It may be beneficial to provide a method and/or structure for predicting/detecting imminent childbirth in order to have better control of the risk of prematurity, in particular a method which allows anticipated/rapid treatment of pregnant women.

Searches for “markers” have been carried out in order to try to develop/identify markers that are of use for predicting preterm birth. For example, the cervical length by ultrasound, the presence of fetal fibronectin of IGFBP-1 and of PAMG-1 in cervical-vaginal secretions have been studied. IGFBP-1 and fibronectin are considered to be markers of cervical maturity. It has been described that a combination of these markers can possibly improve the prediction of a preterm birth [6]. In particular, Eroglu et al. has described the fact that the combination of fibronectin/IGFBP-1 and of the cervical length by ultrasound <25 mm has made it possible to increase the specificity and the positive predictive value for a birth within seven days [1]. However, the usefulness and the implementation of such a test is not relevant or even possible insofar as it requires an ultrasound and an expert in the interpretation of the ultrasound imaging which clearly represents an obstacle to its use [2,3]. Another study carried out by Goepfert describes the fact that a test using IL-6 and fibronectin has shown a correlation [4]. However, a meta-analysis of the usefulness of fibronectin in the prediction, of prematurity within 7-14 days in symptomatic women has shown up a sensitivity of 78-89% and a specificity of 86%, but a lower sensitivity (68-76%) and a comparable specificity (88-89%) in asymptomatic women [5]. In addition, tests using fibronectin have underlined the very low positive predictive value [6] and use/false result limits owing to external factors such as, for example, vaginal bleeding and/or recent unprotected sexual intercourse [7].

Methods using various markers have been envisioned in order to predict a preterm birth. For example Kurkinen-Räty et al. have described the fact that an increase in the cervix, in the IL-6 concentration and in the cervical length by ultrasound measurement appears to be associated with prematurity [8]. However, neither the sensitivity nor the specificity of the tests used in the Kurkinen-Räty study was sufficient to predict preterm birth for taking a good clinical decision.

Furthermore, methods such as cervical elastography or the measurement of fetal respiratory movements have also been the subject of study as potential markers for imminent or preterm birth. Regarding cervical elastography, studies have shown a modification of the mapping before and after maturation of the cervix, but also, between 16 and 24 weeks of amenorrhea, a link between the measurements carried out and a risk of preterm birth. However, this criterion is clearly examiner-dependent with regard to the measurements carried out and there is currently no standard value which makes it possible to draw a conclusion.

Fetal respiratory movements (FRM) have also been studied as a possible marker for the risk of imminent/preterm birth. Indeed, it is known that FRM decrease or cease in the 24 to 36 hours preceding the commencement of labor. Studies have therefore been carried out to measure the predictivity of birth at 48 hours on the basis of FRM. This method has shown predictive results with a sensitivity and specificity similar to the usual methods which do not make it possible to dispense with a clinical examination and/or to avoid the administration of unrequired treatment.

It may be beneficial to provide a method and/or structure for predicting/detecting imminent birth, which does not have the drawbacks/limits of the known methods, in particular a method which makes it possible to obtain results with a sensitivity/specificity/positive and negative predictive value which are compatible with improved treatment of the risk of prematurity.

It may be beneficial to provide a method and/or structure for predicting/detecting preterm birth, the use/results of which do not vary as a function of external factors, for example of any vaginal bleeding and/or of the proximity of sexual intercourse.

Moreover, it is known that there is a great heterogeneity in the clinical signs/tests and in the biochemical mechanisms involved in preterm birth which thus makes the detection/prediction thereof more complex.

It may be beneficial to provide a method and/or structure for predicting/detecting preterm birth, the use/results of which are reliable regardless of the clinical signs and/or biochemical mechanisms involved.

It may be beneficial to address and/or overcome the above issues with the related art by providing a method for the in vitro detection/prediction of imminent birth.

In particular, some embodiments are directed to a method for the in vitro detection/prediction of an imminent birth, including simultaneous search for markers of the group including or consisting of intact insulin-like growth factor binding protein 1 (IGFBP-1 A), of total insulin-like growth factor binding protein 1 (including the intact form and/or an N-terminal fragment) (IGFBP-1 B) and of interleukin 6 (IL-6); an imminent birth, within a predefined period, for example of between 2 and 14 days, is detected/predicted when at least two of the markers of the group including or consisting of insulin-like growth factor binding protein 1 A (IGFBP-1 A), of insulin-like growth factor binding protein 1 B (IGFBP-1 B) and of interleukin 6 (IL-6) are detected.

Intact insulin-like growth factor binding protein 1 (IGFBP-1 A) is sometimes described by some authors under the name placental protein 12 (PP-12) or placental alpha-microglobulin-1 (PAMG-1).

Advantageously, the applicants have demonstrated that the method according to some embodiments makes it possible to exclude an imminent birth when none or just one of the markers are detected. In other words, the method according to some embodiments makes it possible to conclude with increased reliability that there is an absence of threat of imminent birth, thus making it possible to avoid unnecessary hospitalizations.

Some embodiments are directed to a method for the in vitro detection/prediction of an imminent birth in a sample of vaginal or cervical secretions, including:
a) searching for the presence of IGFBP-1 A, of IGFBP-1 B and of IL-6,
b) assigning a value of IGFBP-1 A (I_{A}) if IGFBP-1 A is present in the sample or of 0 if IGFBP-1 A is not present,
c) assigning a value of IGFBP-1 B (I_{B}) if IGFBP-1 B is present in the sample or of 0 if IGFBP-1 B is not present, the value of I_{B} assigned if IGFBP-1 B is present in the sample being less than or equal to the value of I_{A} assigned when IGFBP-1 A is present in the sample,
d) assigning a value of IL-6 (I₆) if IL-6 is present in the sample or of 0 if IL-6 is not present, the value T assigned when IL-6 is present in the sample being strictly less than the values of I_{A} and I_{B} respectively assigned when IGFBP-1 A or IGFBP-1 B is present in the sample,
e) calculating the score S1 according to the following formula:

S₁=I_{A}+I_{B}+I₆,

a value of S1 greater than or equal to the value of I_{A} or I_{B} assigned when IGFBP-1 A or IGFBP-1 B is present in the sample, indicating an imminent birth.

Some embodiments are directed to a method for the in vitro detection/prediction of an imminent birth in a sample of vaginal or cervical secretions, including:
a) searching for the presence of IGFBP-1 A, of IGFBP-1 B and of
b) assigning a value of IGFBP-1 A (I_{A}) if IGFBP-1 A is present in the sample or of 0 if IGFBP-1 A is not present,
c) assigning a value of IGFBP-1 B (I_{B}) if IGFBP-1 B is present in the sample or of 0 if IGFBP-1 B is not present, the value of in assigned if IGFBP-1 B is present in the sample being less than or equal to the value of I_{A} assigned when IGFBP-1 A is present in the sample,
d) assigning a value of IL-6 (I₆) if IL-6 is present in the sample or of 0 if IL-6 is not present, the value I₆ assigned when IL-6 is present in the sample being strictly less than the values of I_{A} and I_{B} assigned when IGFBP-1 A and IGFBP-1 B are respectively present in the sample,
e) calculating the score S1 according to the following formula:

S₁=I_{A}+I_{B}+I₆

a value of S1 less than or equal to the value of I₆ assigned when IL-6 is present in the sample, indicating an absence of imminent birth.

According to some embodiments, the value of IGFBP-1 A (I_{A}) assigned when it is present in the sample is a natural integer greater than 0, for example equal to 1, 2, 3 or 4.

According to some embodiments, the value of IGFBP-1 A (I_{A}) assigned is greater than or equal to I_{B}. For example, the value of I_{A} can be equal to I_{B}, for example equal to 2, the value of I_{A} can be greater than I_{B}, for example equal to 3, and the value of I_{A} equal to 2.

According to some embodiments, the value of IGFBP-1 B (I_{B}) assigned when it is present in the sample is a natural integer greater than 0, for example 1, 2, 3 or 4.

According to some embodiments, the value of IGFBP-1 B (I_{B}) assigned is less than or equal to I_{A}. For example, the value of I_{B} in can be equal to I_{A}, for example equal to 2.

According to some embodiments, the value of IL-6 (I₆) assigned when it is present in the sample is a natural integer, the value of which is strictly less than the values b, and I_{B} assigned.

Some embodiments are directed to a method for the in vitro detection/prediction of an imminent birth in a sample of vaginal or cervical secretions, including:
a) searching for the presence of IGFBP-1 A, of IGFBP-1 B and of IL-6,
b) assigning a value of IGFBP-1 A (I_{A}) equal to 2 if IGFBP-1 A is present in the sample or of 0 if IGFBP-1 A is not present,
c) assigning a value of IGFBP-1 B (I_{B}) equal to 2 if IGFBP-1 B is present in the sample or of 0 if IGFBP-1 B is not present,
d) assigning a value of IL-6 (I₆) equal to 1 if IL-6 is present in the sample or of 0 if IL-6 is not present,
e) calculating the score S1 according to the following formula:

S₁=I_{A}+I_{B}+I₆

a value of S1 greater than or equal to 2 indicating an imminent birth.

Some embodiments are directed to a method for the in vitro detection/prediction of an imminent birth in a sample of vaginal or cervical secretions, including:
a) searching for the presence of IGFBP-1 A, of IGFBP-1 B and of IL-6,
b) assigning a value of IGFBP-1 A (I_{A}) equal to 2 if IGFBP-1 A is present in the sample or of 0 if IGFBP-1 A is not present,
c) assigning a value of IGFBP-1 B (I_{B}) equal to 2 if IGFBP-1 B is present in the sample or of 0 if IGFBP-1 B is not present,
d) assigning a value of IL-6 (I₆) equal to 1 if IL-6 is present in said sample or of 0 if IL-6 is not present,
e) calculating the score S1 according to the following formula:

S₁=I_{A}+I_{B}+I₆

a value of S1 less than or equal to 1 indicating an absence of imminent birth.

According to some embodiments, the term “imminent birth” is intended to mean a birth within a period of 24 hours to 15 days, for example within a period of 14 days, within a period of 7 days, within a period of 48 hours.

In some embodiments, a vaginal or cervical specimen is also denoted by a sample of vaginal or cervical secretions.

According to some embodiments, the term “vaginal or cervical specimen” is intended to mean, for example, a specimen of vaginal or cervico-vaginal secretions taken at the level of the wall of the vagina of a pregnant woman, at the level of the posterior fornix of the vagina, or at the level of the cervix.

According to some embodiments, the specimen can be obtained by any method known to those of ordinary skill in the art. The specimen can be obtained, for example, by pipetting using a pipette or by rubbing the walls of the vaginal cavity, for example with a sterile swab, the bud of which may be cotton or a synthetic fiber. It may for example be made of polyester.

According to some embodiments, the sample may be a sample taken between the 18th and the 42nd week of amenorrhea, for example between the 22nd and 36th week.

In other words, according to some embodiments, the specimen may be a sample taken between the 18th and the 42nd week of amenorrhea, for example between the 22nd and the 36th week.

According to some embodiments, the term “intact IGFBP-1”, or “native IGFBP-1” or “IGFBP-1 A” is intended to mean insulin-like growth factor binding protein 1 (IGFBP-1) having the following sequence SEQ ID No 1:

(SEQ ID No 1)
MSEVPVARVWLVLLLLTVQVGVTAGAPWQCAPCSAEKLALCPPVSASCSE
VTRSAGCGCCPMCALPLGAACGVATARCARGLSCRALPGEQQPLHALTRG
QGACVQESDASAPHAAEAGSPESPESTEITEEELLDNFHLMAPSEEDHSI
LWDAISTYDGSKALHVTNIKKWKEPCRIELYRVVESLAKAQETSGEEISK
FYLPNCNKNGFYHSRQCETSMDGEAGLCWCVYPWNGKRIPGSPEIRGDPN
CQIYFNVQN,

including the phosphorylated forms of the sequence SEQ ID No 1.

According to some embodiments, the term “intact IGFBP-1”, or “native IGFBP-1” or “IGFBP-1 A” is also intended to mean mature insulin-like growth factor binding protein 1 (IGFBP-1) having the following sequence SEQ ID No 2 or the precursors thereof having the following sequence ID No 1 or No 3:

(SEQ ID No 2)
APCSAEKLALCPPVSASCSEVTRSAGCGCCPMCALPLGAACGVATARCAR
GLSCRALPGEQQPLHALTRGQGACVQESDASAPHAAEAGSPESPESTEIT
EEELLDNFHLMAPSEEDHSILWDAISTYDGSKALHVTNIKKWKEPCRIEL
YRVVESLAKAQETSGEEISKFYLPNCNKNGFYHSRQCETSMDGEAGLCWC
VYPWNGKRIPGSPEIRGDPNCQIYFNVQN,
(SEQ ID No 1)
MSEVPVARVWLVLLLLTVQVGVTAGAPWQCAPCSAEKLALCPPVSASCSE
VTRSAGCGCCPMCALPLGAACGVATARCARGLSCRALPGEQQPLHALTRG
QGACVQESDASAPHAAEAGSPESPESTEITEEELLDNFHLMAPSEEDHSI
LWDAISTYDGSKALHVTNIKKWKEPCRIELYRVVESLAKAQETSGEEISK
FYLPNCNKNGFYHSRQCETSMDGEAGLCWCVYPWNGKRIPGSPEIRGDPN
CQIYFNVQN,
(SEQ ID No 3)
SEVPVARVWLVLLLLTVQVGVTAGAPWQCAPCSAEKLALCPPVSASCSEV
TRSAGCGCCPMCALPLGAACGVATARCARGLSCRALPGEQQPLHALTRGQ
GACVQESDASAPHAAEAGSPESPESTEITEEELLDNFHLMAPSEEDHSIL
WDAISTYDGSKALHVTNIKKWKEPCRIELYRVVESLAKAQETSGEEISKF
YLPNCNKNGFYHSRQCETSMDGEAGLCWCVYPWNGKRIPGSPEIRGDPNC
QIYFNVQN,

including the isoforms and phosphorylated forms of the sequences SEQ ID No 1, 2 and/or 3.

According to some embodiments, the term “IGFBP-1 B” is intended to mean intact IGFBP-1 and/or all or part of an N-terminal fragment of mature insulin-like growth factor binding protein 1 (IGFBP-1) having the following sequence SEQ ID No 4 or the precursors thereof having the following sequence ID No 5 or No 6:

(SEQ ID No 4)
APWQCAPCSAEKLALCPPVSASCSEVIRSACFCGCCPMCALPEGAACGVA
TARCARGLSCRALPGEQQPLHALTRGQGACVQESDASAPHAAEAGSPESP
ESTEITEEELLDNFHLMAPSEEDHSILWDAISTYDGSK,
(SEQ ID No 5)
MSEVPVARVWLVLLLLTVQVGVTAGAPWQCAPCSAEKLALCPPVSASCSE
VTRSAGCGCCPMCALPLGAACGVATARCARGLSCRALPGEQQPLHALTRG
QGACVQESDASAPHAAEAGSPESPESTEITEEELLDNFHLMAPSEEDHSI
LWDAISTYDGSK,
(SEQ ID No 6)
SEVPVARVWLVLLLLTVQVGVTAGAPWQCAPCSAEKLALCPPVSASCSEV
TRSAGCGCCPMCALPLGAACGVATARCARGLSCRALPGEQQPLHALTRGQ
GACVQESDASAPHAAEAGSPESPESTEITEEELLDNFHLMAPSEEDHSIL
WDAISTYDGSK,

including the isoforms and phosphorylated forms of the sequences SEQ ID No 4, 5 and/or 6.

In other words, the intact or native insulin-like growth factor binding protein (IGFBP-1) can be independently denoted IGFBP-1N or IGFBP-1 A.

In other words, intact IGFBP-1 and/or an N-terminal fragment of insulin-like growth factor binding, protein 1 (IGFBP-1) can be independently denoted IGFBP-1 T or IGFBP-1 B.

According to some embodiments, the antibodies directed against IGFBP-1 A (anti-IGFBP-1 A antibodies) can be chosen, for example, from the group including the antibodies described in the documents Rutanen et al. Biochem Biophys Res Commun 1988; 152: 208 [9] and in Pekonen et al., J immunoassay 1989; 10: 325-337 [10], the anti-IGFBP-1 antibodies sold by the company Hytest (catalog number 4IG8; clone G5F8 and clone C7B9).

Advantageously, the IGFBP-1 A detection threshold in the sample can be chosen; for example, IGFBP-1 A can be considered to be present in the sample when its concentration is greater than or equal to 40 ng/ml of IGFBP-1.

According to some embodiments, the antibodies directed against IGFBP-1 B (anti-IGFBP-1 B antibodies) can be the mixture of antibodies sold by the company Hytest, catalog number 4I52; clone G2; catalog number 4IG8; clone G5F8 and clone C7B9.

The applicants have noted that the mixtures of antibodies above make it possible to distinguish IGFBP-1 A from IGFBP-1 B since the binding sites of the antibodies are located at different locations on the protein. For example, the antibody with catalog number 4I52 (Hytest); clone G2 binds at the N-terminal level of IGFBP-1.

According to some embodiments, the pH of the solution including the anti-IGFPB-1B antibodies can be 7.4.

Advantageously, the IGFBP-1 B detection threshold in the sample can be chosen; for example, IGFBP-1 B can be considered to be present in the sample when its concentration is greater than or equal to 20 ng/ml of intact IGFBP-1 and/or 10 ng/ml of the N-Ter fragment of IGFBP-1.

According to some embodiments, the antibodies directed against interleukin 6 (anti-IL-6 antibodies) can be chosen, for example, from the group including the antibodies sold by Diaclone; catalog number 855.050.005, clone B-E8 or B-E4.

According to some embodiments, the pH of the solution including the anti-IL-6 antibodies can be 7.4.

Advantageously, the IL-6 detection threshold in the sample can be chosen; for example, IL-6 can be considered to be present in the sample when its concentration is greater than or equal to 0.25 ng/ml.

Advantageously, according to some embodiments, the concentration of each antibody and/or the pH of each solution including each antibody can make it possible to modulate the detection threshold for the IGFBP-1 A, IGFBP-1 B or interleukin 6.

According to some embodiments, the antibodies directed against IGFBP-1 A, IGFBP-1 B or interleukin (can be independently advantageously in dry form.

According to some embodiments, the antibodies directed against IGFBP-1 A, IGFBP-1 B and IL-6 can be labeled or non-labeled. The labeling is set out below. Advantageously, the anti-IGFBP-1 A antibodies used for capture and the labeled anti-IGFBP-1 A antibodies can be directed against different epitopes, the anti-IGFBP-1 B antibodies used for capture and the labeled anti-IGFBP-1 B antibodies can be directed against different epitopes, and the anti-IL-6 antibodies used for capture and the labeled anti-IL-6 antibodies can be directed against different epitopes.

According to some embodiments, the antibodies directed against IGFBP-1 A, IGFBP-1 B or interleukin 6 can be independently labeled by any methods known to those of ordinary skill in the art. This may, for example, be a chemical molecule, for example a fluorescent label, for example rhodamine or fluorescein isothiocyanate (FITC), an enzyme, for example horseradish peroxidase, alkaline phosphatase, ⋅-galactosidase, glucose 6-phosphate dehydrogenase, which is revealed in the presence of a specific substrate, for example TMB (or 3,3′,5,5′-tetramethylbenzidine), 4-methylumbelliferyl phosphate, colored nanoparticles, for example latex or polystyrene particles, colloidal gold particles, liposomes, carbon or selenium particles, fluorescent nanoparticles containing, for example, lanthanide (europium for example) chelates, or magnetic nanoparticles, radioactive molecules, for example iodine 125, iodine 133, tritium.

According to some embodiments, the labeling can be different according to the specificity of the antibody. For example, the anti-IGFBP-1 A antibody can be labeled with a label that is different than that of the anti-IGFBP-1 B antibody, and also different than that of the anti-IL-6 antibody. Advantageously, the difference in labeling makes it possible to detect whether only IGFBP-1 A, only IGFBP-1 B or only interleukin 6, and/or any combination of proteins, are present in the sample by virtue of the difference in labeling. The different labelings make it possible to detect IGFBP-1 A, l'IGFBP-1 B and interleukin 6, in a distinct manner.

According to some embodiments, the antibodies can be pre-bound to a solid support. For example, the antibodies directed against IGFBP-1 A, IGFBP-1 B or interleukin 6 can be bound to a membrane, for example a nitrocellulose membrane, the bottom of a multiwell plate, an absorbent paper, a glass fiber support and/or any surface known to those of ordinary skill in the art which makes it possible to bind at least one antibody.

The controlling of the amount of antibody bound advantageously makes it possible to adjust the detection threshold for IGFBP-1 A, IGFBP-1 B and interleukin 6. Specifically, the higher the amount bound, the lower the detection threshold.

In other words, according to some embodiments, the antibodies can be pre-bound to a support, for example by deposition or by chemical coupling.

When the antibodies are bound by deposition, they can be deposited, for example, by sprinkling, onto a common or distinct surface, of anti-IGFBP-1 A, anti-IGFBP-1 B and anti-interleukin 6 antibodies as defined previously, then drying, for example via the method described in Beer et al., Qualification of cellulose nitrate membranes for lateral-flow assays, IVD technology, January 2002. [11].

When the antibodies are bound by chemical coupling, they can be bound, for example, by the method described in Beer et al., Qualification of cellulose nitrate membranes for lateral-flow assays, IVD technology, January 2002. [11]

According to some embodiments, the method can also include a preliminary step of diluting the vaginal or cervical specimen in a diluting solution. The diluting solution can be any solution known to those of ordinary skill in the art for diluting these specimens. It may for example be a physiological solution, or a buffer solution. It may for example also be a solution including 50 mM Na₂HPO₄ (S7907, Sigma Aldrich 1% BSA (1035-70, ID Bio), 0.05% triton X-100, 0.55% Tween 20 (P7949, Sigma Aldrich) (pH 7.4).

According to some embodiments, the pH of the diluting solution can be from 7 to 8.

According to some embodiments, the ionic strength of the diluting solution can be from 10 to 1000 mM, preferably from 50 to 200 mM.

According to some embodiments, when the specimen of vaginal or cervical secretions collected by a swab including the bud, it can be prediluted in a volume of buffer solution of from 0.5 to 2 ml, for example 1 ml.

According to some embodiments, the method can include a revealing step.

According to some embodiments, the revealing step can be carried out by any method known to those of ordinary skill in the art. It can for example be a radioimmunological (RIA), immunoradiometric (IRMA), enzyme immunological (EIA), immunofluorometric (IFMA), time-resolved immunofluorometric, luminoimmunological (LIA) or immunomagnetic method, or a method by immunochromatography or immunofiltration using colored (latex, polystyrene or colloidal) particles. Some of these methods can be carried out as described in Anne Harwood Peruski et al., Immunological Methods for Detection and Identification of Infectious Disease and Biological Warfare Agents. Clinical and diagnostic laboratory immunology, July 2003, p. 506-513. [12]

According to some embodiments, the revealing can for example be direct or indirect.

When the revealing is indirect or by competition, use may for example be made of the IGFBP-1 A. IGFBP-1 B or IL-6 antigen winch is labeled, for example, with particles, as described previously.

When the revealing is direct (sandwich system), use may for example be made of a capture antibody and a labeled, second antibody directed against IGFBP-1 A, a capture antibody and a labeled second antibody directed against IGFBP-1 B and a capture antibody and a labeled second antibody directed against IL-6 as described previously. Advantageously, the capture antibody and the labeled antibody are directed against different epitopes so as to form a pair that can be used in the sandwich system.

Preferably, the revealing is direct.

Advantageously, the intensity of the signal of the label depends on the amount and/or on the concentration of IGFBP-1 A, of IGFBP-1 B and/or of IL-6 present in the specimen.

Depending on the label used, the revealing may be visual or carried out using a device.

When the label is a visible colored label as described previously, the revealing is advantageously visual. When the label is, for example, a radioactive label as described previously, the revealing will be carried out, for example, using a device for measuring radioactivity, for example a Geiger counter.

According to some embodiments, the method include an adjustment of at least one of the parameters chosen from the volume of the diluting buffer medium, the pH of the buffer medium, the ionic strength of the buffer medium, the adjustment being common for IGFBP-1 A, IGFBP-1 B and IL-6.

According to some embodiments, the method can include an adjustment of at least one of the parameters chosen from the concentration of the capture antibodies and/or the concentration of the labeled antibodies, the adjustment being specific to IGFBP-1 A, IGFBP-1 B or IL-6.

According to some embodiments, the method advantageously makes it possible, by virtue of the complementarily of the labels, to detect/predict an imminent birth in a pregnant woman. In particular, the method according to some embodiments advantageously makes it possible, depending on the presence or absence of the labels in the sample, to identify a subject for whom there is a risk of giving birth within 14 days, advantageously within 7 days.

Advantageously, the applicants have demonstrated that the method according to some embodiments allows the detection/prediction of a birth, in particular within 7 days, with a specificity of greater than 93% and a sensitivity of greater than 87%.

Some embodiments advantageously make it possible to decrease the probability of obtaining false-negative results and also false-positive results and more particularly in the presence of triple-negative or triple-positive results with regard to the three parameters included in the method. Thus, the result obtained makes it possible to conclude that birth is imminent without any additional clinical test.

In addition, some embodiments advantageously make it possible to detect a preterm birth regardless of the stage of the pregnancy.

In addition, the applicants have advantageously shown that, when the three markers, namely intact insulin-like growth factor binding protein 1 (IGFBP-1 A), total insulin-like growth factor binding protein 1 (including the intact form and/or an N-terminal fragment) (IGFBP-1 B) and interleukin 6 (IL-6), are simultaneously detected in or absent from the sample/specimen of vaginal or cervical secretions, the positive and negative predictivity are respectively increased. The applicants have advantageously shown that, when none of the markers is detected, the negative predictive value of birth in the next 2 days is 100%, in the next 7 days or 14 days is 98%. The applicants have also advantageously demonstrated that, when all the markers are detected, the positive predictive value of birth in the next 7 days or 14 days is greater than 95.8%.

Advantageously, the applicants have shown that, when the score S1 is greater than or equal to I_{A} I_{A} being equal to the value assigned when IGFBP-1 A is present in the sample, for example greater than or equal to 2, the negative predictive value is greater than 96.8%. In particular, the applicants have shown that, regardless of the imminent nature of the birth, for examples less than 2 days, less than 7 days and/or less than 14 days, when the score S1 is greater than or equal to I_{A}, I_{A} being equal to the value assigned when IGFBP-1 A is present in the sample, for example greater than or equal to 2, the greater negative predictive value is greater than 96.8%, ranging as far as 98.4%.

Advantageously, the applicants have shown that when the score S1 is equal less than or equal to the value I₆ to assigned when IL-6 is present in the sample, for example equal to 0 or 1, the probability of giving birth in the next 1.4 days is less than 8.3% to zero.

Advantageously, the applicants have also shown that, when the score S1 is between I_{A} and (I_{A}+I_{B}), the values of I_{A} and I_{B} being equal to the values assigned when IGFBP-1 A. and IGFBP-1 B are present in the sample, for example included from 2 to 4, the probability of giving birth within 14 days is greater than 90%.

Advantageously, the applicants have also shown that, when the score S1 is equal to the sum of the values assigned when IL-6, IGFBP-1 A and IGFBP-1 B 5 are present in the sample, for example equal to 5, the probability of giving birth within 7 days is greater than 95%.

Advantageously, the applicants have also shown that a score S1 equal to 0, that is to say for which none of the markers is detected at a negative predictive value tier giving birth in the next 2 days of 100%, in the next 7 days or 14 clays of 98%. In other words, the applicants have clearly demonstrated that the method of some embodiments, in particular as a function of the score S1 obtained, makes it possible to reduce or even to totally exclude the false-negative results, advantageously making it possible to exclude with certainty an imminent birth.

Advantageously, the applicants have also shown that a score S1 equal to the swat of the values assigned when IL-6, IGFBP-1 A and IGFBP-1 B 5 are present in the sample, for example equal to 5, that is to say for which all the markers are detected at a positive predictive value of birth in the next 7 days or 14 days greater than 95.8%. In other words, the applicants have clearly demonstrated that the method of some embodiments, in particular as a function of the score S1 obtained, makes it possible to conclude that birth is imminent with a very high predictive value.

The method according to some embodiments can also include a step of measuring the cervical length on an image obtained by ultrasound.

When the cervical length is measured on an image obtained by ultrasound, a cervical length of between 20 and 30 mm, preferably less than 25 mm, is considered as increasing the risk of imminent birth.

The method according to some embodiments can also include a step of measuring the cervical dilation on an image obtained by ultrasound.

When the cervical dilation measured on an image obtained by ultrasound is greater than or equal to 3 cm it is considered as increasing the risk of imminent birth.

According to some embodiments, the ultrasound image can be obtained by any device known to those of ordinary skill in the art. It may for example be a commercially available device, for example sold by the company GE Healthcare, Philips.

According to some embodiments, the ultrasound image can be obtained before or after the step of searching for IGFBP-1 A, IGFBP-1 B and/or IL-6. For example, the ultrasound image can be obtained 1 to 12 hours before the searching step or from 0.5 to 12 hours after the searching step.

Advantageously or preferably, the measurement of the cervical dilation is carried out after the step of searching for IGFBP-1 A, IGFBP-1 B and/or IL-6.

Advantageously, the method according to some embodiments can also be used for detecting/measuring a risk of premature rupture of the fetal membranes.

In addition, the method according to some embodiments also advantageously makes it possible to detect/measure a risk of premature rupture of the fetal membranes. In particular, since a preterm birth involves a rupture of the fetal membrane, the prediction/detection of an imminent birth also makes it possible to detect a risk of premature rupture of the fetal membranes.

In particular, in this embodiment, when IGFBP-1 A and/or IGFBP-1 B and/or IL-6 are detected in the sample, this can in particular imply a risk of rupture of fetal membranes.

Advantageously, the method according to some embodiments can also be used for determining the optimal period for triggering birth.

Some embodiments are directed to a device, for example an immunochromatographic device, for implementing the method, including:
a sample-depositing zone (1),
a zone (2) including labeled anti-IGFBP-1 A antibodies and labeled anti-IGFBP-1 B antibodies and labeled anti-IL-6 antibodies,
a revealing zone (3) including capture antibodies directed against IGFBP-1 A,
a revealing zone (4) including capture antibodies directed against IGFBP-1 B,
a revealing zone (5) comprising capture antibodies directed against IL-6,
optionally, a fluid absorption zone (6).

According to some embodiments, the depositing zone (1) can be a zone suitable for the application or the reception of the specimen. This zone may be in any form known to those of ordinary skill in the art, for example a reservoir, a cup, a well, a wick or a flat surface.

According to some embodiments, the depositing zone (1) may be mobile an or linked to any one of the zones (2) to (5), When the zone (1) is mobile, it may, for example, be used to take the specimen and be applied over one or all of the zones (2) to (5). When the zone (1) is linked to one of the zones (2) to (5), the zone can be dipped directly into a container including the specimen and/or the specimen can be applied to this zone.

According to some embodiments, the materials of the zones (1) to (5) can be identical or different. The materials can be any material known to those of ordinary skill in the art, for example a material chosen from the group including absorbent paper, cotton wool, cellulose, glass fiber, cellulose fiber, a nitrocellulose membrane. They may also be the abovementioned materials which have undergone one or more prior treatments, for example by bringing the material into contact with a solution. The solution may include one or more chemical components, for example sodium tetraborate and/or sodium phosphate supplemented with detergent such as tween 20 and/or triton X-100 and/or with proteins such as bovine serum albumin or casein. The solution can have a pH of between 6 and 10, preferably between 7 and 9. Advantageously, bringing the solution into contact with the material can make it possible to modify the surface properties of the material, for example to make it hydrophilic, to improve the migration of the sample, to improve the binding and the stability of the conjugate, to have an impact on the performance levels of the test.

Advantageously, the depositing zone (1) can be chosen from the group including an absorbent paper, a glass fiber support and cellulose fiber.

Advantageously, the zone (2) including labeled anti-IGFBP-1 A antibodies, labeled anti-IGFBP-1 B antibodies and labeled anti-IL-6 antibodies may be glass fiber and cellulose fiber.

Advantageously, the zones 1 and 2 can consist of the same material chosen from the group including an absorbent paper, a glass fiber support and cellulose fiber.

Advantageously, the revealing zones (3) to (5) can be a nitrocellulose membrane.

According to some embodiments, the capture antibodies directed against IGFBP-1 A, directed against IGFBP-1 B and directed against IL-6 can be, for example, directly bound to the support of the zone, for example by chemical coupling and/or by deposition as described previously.

According to some embodiments, when the zones (3) to (5) are located on one and the same support, the zones for binding the IGFBP-1 A, IGFBP-1 B and IL-6 capture antibodies are preferentially different than one another. They may for example be three parallel strips each including a different capture antibody.

According to some embodiments, the various abovementioned zones (1) to (6) can be bound to a solid support, for example to laminated cards, for example which can be cut up into strips, and which can be included in a container including one or more orifice(s) at the level of the zones (3) to (5) allowing visualization of the result and an orifice at the level of the sample depositing zone (1) allowing deposition of the sample.

According to some embodiments, the various abovementioned zones (1) to (6) can be bound to various solid supports, for example to laminated cards, for example in strips, and can be included in various containers. For example, the various zones can be bound to three supports each including a zone (1), a zone (2), a zone chosen from group including the zones (3), (4) and (5), and a zone (6). Each support or container can include an orifice at the level of the zones (3) to (5) allowing, visualization of the result and a orifice at the level of the sample-depositing zone (1) allowing deposition of the sample.

According to some embodiments, the device can further include an absorption zone (6). According to some embodiments, the absorption zone (6) can be any absorbent solid support known to those of ordinary skill in the art. It can for example be an absorbent blotting paper, a sponge, a cotton, felt or a synthetic textile.

Advantageously, when the device includes the various abovementioned zones (1) to (5) bound to a solid support, the supports of the zones (1) and (2) overlap at one of their ends, and the other end of the zone (2) overlaps with one end of the material including the zones (3) to (5). The overlapping of the various zones (1) to (3-5) advantageously makes it possible, when the specimen is applied and/or when the free end of the zone (1) is immersed in the specimen, for the specimen to migrate in the various zones via capillary action. Preferably, the zones (1) and (2) are arranged on one and the same support. Preferably, the absorption zone (6) overlaps with the free end of the zone (5). Thus, the zone (6) allows accelerated migration of the specimen through the various zones of the device. Advantageously, the zone (6) makes it possible to absorb the excess liquid of the specimen.

Advantageously, when the device includes various zones that can be bound to three supports each includes a zone (1), a zone (2) and a zone chosen from the group including the zones (3), (4) and (5). On one support, the zones (1) and (2) overlap at one of their ends, and the other end of the zone (2) overlaps with one end of the zone (3); on a second support, the zones (1) and (2) overlap at one of their ends, and the other end of the zone (2) overlaps with one end of the zone (4); and on a third support the zones (1) and (2) overlap at one of their ends, and the other end of the zone (2) overlaps with, one end of the zone (5). Preferably, an absorption zone (6) overlaps independently with the free end of the zone (3), (4) and/or (5). Thus, the zone (6) allows accelerated migration of the specimen through the various zones of the device. A Advantageously, the zone (y) makes it possible to absorb the excess liquid of the specimen.

The overlapping of the various zones (1) to (5) advantageously makes it possible, when the specimen is applied and/or when the free end of the zone (1) is immersed in the specimen, for the specimen to migrate in the various zones via capillary action.

According to some embodiments, the various zones (1) to (6) can be independently covered with a protective film. T his may, for example, be a plastic film, fir example a polyvinyl chloride (PVC) film, or a biodegradable film, for example a polycaprolactone (PCL), polyvinyl alcohol (PVA) or polylactic acid (PLA) film.

The film advantageously makes it possible to independently protect the various zones of the device of some embodiments.

According to some embodiments, the film may be a single film or several films independently covering one of the zones (1) to (6).

According to some embodiments, the film or films may partially cover the device of some embodiments, thus leaving certain zones uncovered. According to some embodiments, the film or films may be a detachable film or detachable films which may be removed before the use of the device, According to some embodiments, the film may include an inscription at its surface which advantageously makes it possible to identify the device.

Some embodiments are directed to a kit for implementing the method of some embodiments, including:
anti-IGFBP-1 A, anti IGFBP-1 B and anti-IL-6 capture antibodies as defined previously,
labeled anti-IGFBP-1 A, anti IGFBP-1 B and anti-IL-6 antibodies as defined previously.

According to some embodiments, the kit may also include a sampling method, for example a swab, as defined previously.

According to some embodiments, the kit may also include a diluting method, for example a diluting buffer solution contained in a bottle, preferably of dropper type, as defined previously.

According to some embodiments, the kit may also include a solid support to which capture antibodies as defined previously can be bound.

Other advantages may further emerge to those of ordinary skill in the art on reading the examples below, illustrated by the appended figures, given by way of illustration.

### BRIEF DESCRIPTION OF THE FIGURES

FIG. 1A represents a diagram of a device for detecting interleukin 6 (IL-6), and the support B including the device. In this figure, (1) corresponds to a plastic support with adhesive which is 6 cm high, (2) a nitrocellulose membrane: 2.5 cm high, (3) a conjugate pad sold by the company Ahlstrom #6613 which has been treated; 1 cm, (4) a sample pad: sold by the company Ahlstrom #8964 which has been treated; 1.6 cm, (5) an absorbent pad: sold by the company Ahlstrom #222; 1.6 cm, (6) a goat anti-mouse IgG antibody, (7) an anti-human IL-6 antibody, (8) a labeled anti-human IL-6 antibody.

FIG. 2A represents a diagram of a device for detecting, IGFBP-1 A, and the support B including the device. In this figure, (9) corresponds to a plastic support with adhesive, 6 cm high, (10) a nitrocellulose membrane: 2.5 cm high, (11) a sample pad: sold by the company Ahlstrom #8%4 which has been treated: 2.3 cm, (12) an absorbent pact: sold by the company Ahlstrom #222; 1.6 cm, (13) a goat anti-mouse IgG antibody, (14) a mouse anti human IGFBP-1 antibody, (15) a labeled anti-human IGFBP-1 antibody.

FIG. 3A represents a diagram of a device for detecting IGFBP-1 B, and the support B including the device. In this figure, (9) corresponds to a plastic support with adhesive, 6 cm high, (10) a nitrocellulose membrane: 2.5 cm high, (11) a sample pad: sold by the company Ahlstrom #8964 which has been treated; 2.3 cm, (12) an absorbent pad: sold by the company Ahlstrom #222; 1.6 cm, (13) a goat anti-mouse IgG antibody. (14) a mouse anti human IGFBP-1 antibody, (16) a mixture of labeled anti-human N-terminal IGFBP-1 and anti human IGFBP-1 antibodies.

FIG. 4 represents a scheme of a device for detecting IL-6, IGFBP-1 A, IGFBP-1 B, fibronectin A (FNA) and fibronectin B (FNB) and the swab used for taking the sample.

FIG. 5 represents a diagram concerning the study of the prediction/detection of preterm birth.

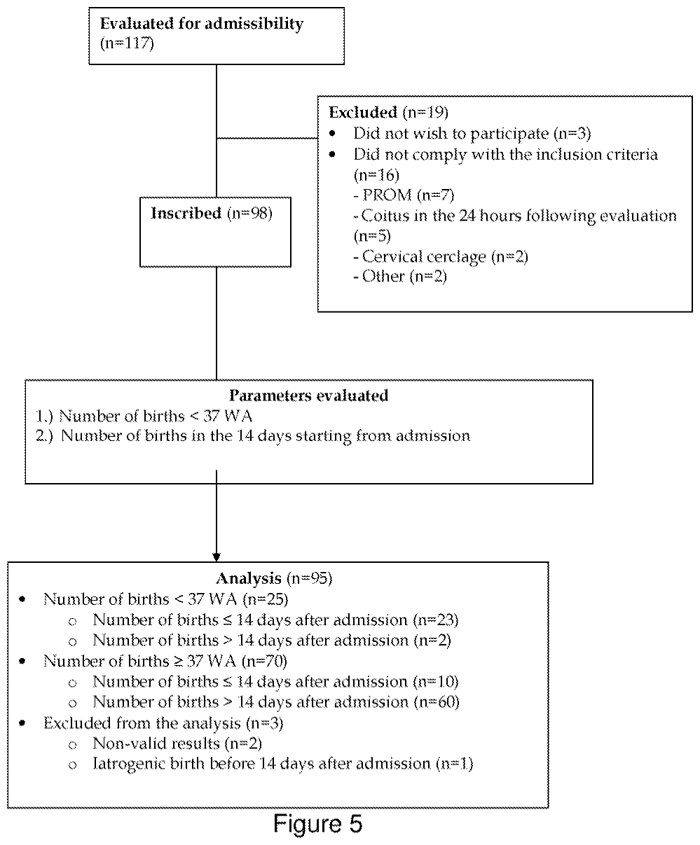

FIG. 6 represents the support including devices for detecting, respectively, IL-6, IGFBP-1 A and IGFBP-1 B.

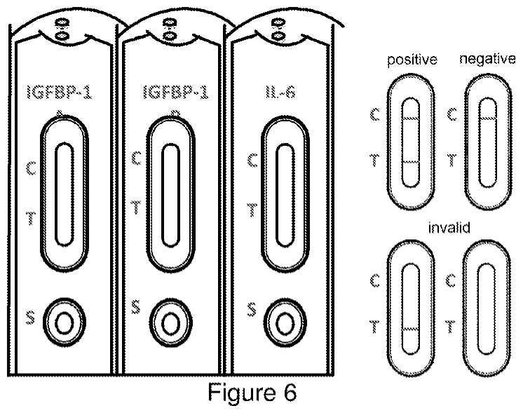

FIG. 7 represents a diagram of a device for detecting IGFBP-1 A, IGFBP-1 B and IL-6. In this figure, (9) corresponds to a plastic support with adhesive, 6 cm high, (10) a nitrocellulose membrane: 2.5 cm high, (11) a sample pad: sold by the company Ahlstrom #8964 which has been treated; 2.3 cm, (12) an absorbent pad: sold by the company Ahlstrom #222; 1.6 cut, (13) a goat anti-mouse IgG antibody, (14) a mouse anti-human IGFBP-1 antibody, (7) an anti-human IL-6 antibody, (17) an anti-human N-terminal IGFBP-1 antibody, (18) a mixture of labeled anti-human IGFBP-1 and anti-human IL-6 antibodies.

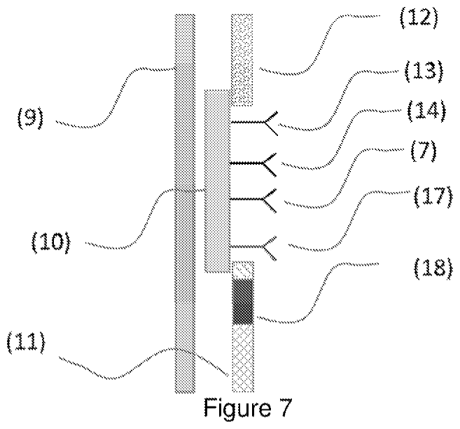

### EXAMPLES

### Example 1: Immunochromatographic Device for Evaluating the Risk of Imminent Birth

The device is composed of three distinct strips: a strip detecting IGFBP-1 A, a strip detecting IGFBP-1 B and a strip detecting IL-6.

### Preparation of the IL-6 Strip

The strips were prepared from laminated cards of 30 cm×6 cm (CNPC-SS12, MDI (registered trademark)) including or consisting of a plastic support covered with a layer of adhesive on which the nitrocellulose membrane, the absorbent paper and the glass fibers are assembled.

Using an automated reagent dispenser (Isoflow Dispenser, Imagene Technology Inc. (registered trade mark)), goat anti-mouse IgG antibodies (ABGAM-0500, Arista Biologicals (registered trade mark)) diluted to 0.5 mg/ml in a PBS buffer and also anti-IL-6 antibodies (catalog number 855.050.005, clone B-E8, Diaclone (registered trade mark)) diluted to 1 mg/ml in a PBS buffer were deposited, with a flow rate of 1 μl/cm, in the form of parallel lines 5 mm apart and having a width of 1 to 2 min each, on a nitrocellulose membrane, the width of which is 25 mm.

After deposition of these searching methods, these cards were then placed for 30 minutes in an oven at 37° C. under an atmosphere with a controlled humidity of less than 30% in order to dry them.

The conjugate mixture, that is to say a solution including labeled antibodies, was prepared from the anti-IL-6 antibodies (catalog number 879.030.002, clone B-E4, Diaclone (registered trademark)) coupled beforehand to colloidal gold particles according to the following protocol:

1.0 ml of 1% gold chloride solution (G4022, Sigma Aldrich) was added to 100 ml of distilled water. The water was heated to boiling point and 2.5 ml of 1% sodium citrate solution (S1804, Sigma Aldrich) were added. The solution became colorless and then turned purple. After one minute, the solution was heated until it became cherry red. 9.5 ml of 1% sodium citrate (S1804, Sigma Aldrich) were added. The water was heated to boiling point and 8.6 ml of 1% gold chloride solution (G4022, Sigma Aldrich) were added. The solution became dark blue, virtually black, then purple, and then was left to cool at ambient temperature.

The pH of 10 ml of the previously obtained purple colloidal gold solution was adjusted, using a 0.2 M potassium carbonate solution (269619, Sigma Aldrich), until a pH of 6.3 was reached. 200 μg of anti-IL-6 were added to 1 ml of distilled water and then 10 ml of purple colloidal gold solution with the pH adjusted to 6.3 were rapidly added. This tube was placed on a circular mixer (multidisk rotator 5-50 rpm, Sobinco (registered trademark)) for 20 minutes. 1.5 ml of Gold stabilization Buffer (Artron Bioresearch (registered trademark)) were added. The 2 tubes were centrifuged (5804, Eppendorf (registered trademark)) at 4000 ref for 30 minutes. The supernatant was suctioned off using a vacuum pump (159600, Brand (registered trademark)) and the pellet was taken up with 1 ml of resuspension buffer (pH 5.0) containing 20 mM Tris Base (26-128-3094, Euromedex (registered trademark)), 50 mM NaCl (S7653, Sigma Aldrich (registered trademark)), 0.2% BSA (1035-70, ID Bio (registered trademark)), 10% sucrose (S8501, Sigma Aldrich), 5% trehalose (T9531, Sigma Aldrich (registered trademark)).

800 μl of the saturating solution ((pH. 74) 50 mM Na₂HPO₄ (S7907, Sigma Aldrich (registered trademark)), 1% of bovine serum albumin (BSA) (1035-70, ID Bio), 20% of sucrose (S8501, Sigma Aldrich) and 5% of trehalose (T9531, Sigma Aldrich (registered trademark))) were added to 200 μl of IL-6 conjugate solution. The resulting mixture was stirred for 20 minutes at ambient temperature on the circular mixer adapted for 1.5 ml tubes.

The mixture of antibodies coupled to the colloidal gold particles was then sprayed with a flow rate of 4 μl/cm onto glass fiber sheets (conjugate pad zone) having the dimensions 30 cm×1 cm (#6613 Ahlstrom, MAPDS-200, Arista Biologicals (registered trademark)) pretreated with a solution of 0.5% of polyvinyl alcohol P8136, Sigma Aldrich (registered trademark)), 0.1% of triton X-100 (X-100, Sigma Aldrich (registered trademark)), 0.5% of bovine serum albumin (BSA) (1035-70, ID Bio), 50 mM Na₂HPO₄ (S7907, Sigma Aldrich (registered trademark)), pH 7,4.

The binding of this mixture of conjugates to the glass fiber sheet was carried out with the same Isoflow Dispenser (registered trademark) apparatus by complying with the manufacturer's indications. After spraying, the glass fiber sheets are dried for 30 minutes in an oven at 37° C. under an atmosphere at a controlled humidity of less than 30% in order to dry them.

The cards were assembled by adhesively bonding the conjugate-impregnated glass fiber onto the lower adhesive part of the card, making the glass fiber sheet overlap by 1 to 3 mm on the nitrocellulose membrane. A second glass fiber paper (sample pad zone) having dimensions of 30 cm×1.6 cm (#8964 Ahlstrom, MAPDS-0300, Arista Biologicals (registered trademark)) pretreated with a solution of 0.1 M of B₄Na₂O₇ (B3545, Sigma Aldrich (registered trademark)), 1% triton X-100 (X-100, Sigma Aldrich (registered trademark)), pH 8.4, is adhesively bonded in the lower adhesive part of the card while overlapping the conjugate-impregnated glass fiber sheet by 1 to 3 mm.

In the same way, a paper with high absorption capacity (absorbent pad zone) (absorbent pad, Ahlstrom 222, MAPDS-0100, Arista Biologicals (registered trademark)) having dimensions of 30 cm×1.6 cm was placed on the upper part of the card and overlaps the nitrocellulose membrane by 1 to 3 mm in order to be able to create a migration flow.

The card thus assembled was cut up into strips of 4 mm wide using a guillotine (model CM4000, Biodot, (registered trademark)).

The strips were then placed in a plastic support called a cassette (MK001, D2 Technologies (registered trademark)).

The cassettes were then closed using a clipping device (Closure-I, A-Point Technologies (registered trademark)).

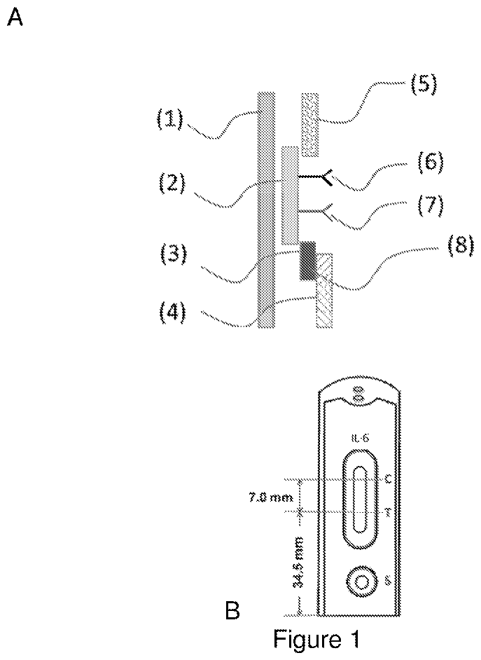

FIG. 1 represents a strip and a support including the strip for detecting IL-6.

### Preparation of the IGFBP-1 A Strip

The strips were prepared from laminated cards of 30 cm×6 cm (CNPC-SS12, MDI (registered trademark)) including or consisting of a plastic support covered with a layer of adhesive on which the nitrocellulose membrane, the absorbent paper and the glass fiber were assembled.

Using an automated reagent dispenser (Isoflow Dispenser, Imogene Technology Inc. (registered trademark)), goat anti-mouse IgG antibodies (ABGAM-0500, Arista Biologicals (registered trademark)) diluted to 0.5 mg/ml in a PBS buffer and also anti-IGFBP-1 antibodies (catalog number 4IG8, clone C7B9, Hytest (registered trademark)) diluted to 1 mg/ml in a PBS buffer were deposited, with a flow rate of 1 μl/cm, in the form of parallel lines 5 mm apart and from 1 to 2 mm wide each, on a nitrocellulose membrane, the width of which is 25 mm.

After deposition of these searching methods, these cards were then placed for 30 minutes in an oven at 37° C. under an atmosphere at a controlled humidity of less than 30% in order to dry them.

The conjugate mixture, that is to say a solution including labeled antibodies, was prepared from the anti-IGFBP-1 antibody (catalog number 4IG8, clone G5F8, Hytest (registered trademark)) coupled beforehand to colloidal gold particles according to the following protocol:

1.0 ml of 1% gold chloride solution (G4022, Sigma Aldrich) was added to 100 ml of distilled water. The water was heated to boiling point and 2.5 ml of 1% sodium citrate solution (S1804, Sigma Aldrich) were added. The solution became colorless and then turned purple. After one minute, the solution was heated until it became cherry red. 9.5 ml of 1% sodium citrate. (S1804, Sigma Aldrich) were added. The water was heated to boiling point and 8.6 ml of 1% gold chloride solution (G4022, Sigma Aldrich) were added. The solution became dark blue, virtually black, then purple and was then left to cool to ambient temperature.

The pH of 10 ml of the purple colloidal gold solution previously obtained was adjusted, using a 0.2 M potassium carbonate solution (269619, Sigma Aldrich), until a pH of 7.8 was reached. 200 μg of anti-AIGFBP-1 clone F8 were added to 1 ml of distilled water and then 10 ml of purple colloidal gold solution at the pH adjusted to 7.8 were rapidly added. These tubes were placed on a circular mixer (multidisk rotator 5-50 rpm, Sobinco (registered trademark)) for 20 minutes. 1.5 ml of gold stabilization buffer (Artron Bioresearch (registered trademark)) were added. The 2 tubes were centrifuged (5804, Eppendorf (registered trademark)) at 4000 rcf for 30 minutes. The supernatant was suctioned off using a vacuum pump (159600, Brand (registered trademark)) and the pellet was taken up with 1 ml of resuspension buffer (pH 8.0) containing 20 mM Tris Base (26-128-3094, Euromedex (registered trademark)), 50 mM NaCl (S7653, Sigma Aldrich (registered trademark)), 0.2°BSA (1035-70, ID Bio (registered trademark)), 10% sucrose (S8501, Sigma Aldrich), 5% trehalose (T9531, Sigma Aldrich (registered trademark)).

800 μl of the saturating solution ((pH 7.4) 50 mM Na₂HPO₄ (S7907, Sigma Aldrich (registered trademark)), 1% of bovine serum albumin (BSA) (1035-70, ID Bio), 20% of sucrose (S8501, Sigma Aldrich) and 5% of trehalose (T9531, Sigma Aldrich (registered trademark))) was added to 200·1 of solution of IGFBP-1 conjugate, clone F8. The mixture thus obtained was stirred for 20 minutes at ambient temperature on the circular mixer adapted for 1.5 ml tubes.

The mixture of antibodies coupled to the colloidal gold particles was then sprayed, with a flow rate of 4 μl/cm, onto glass fiber sheets (sample/conjugate pad zone) having dimensions of 30 cm×3.2 cm (#8964 Ahlstrom, MAPDS-0300, Arista Biologicals (registered trademark)) pretreated with a solution of 0.1 M B₄Na₂O₇ (B3545, Sigma Aldrich (registered trademark)), 1% triton X-100 (X-100, Sigma Aldrich (registered trademark)), pH 8.4.

The binding of this conjugate mixture to the glass fiber sheet was carried out with the same Isoflow Dispenser (registered trademark) apparatus while complying with the manufacturer's indications. After spraying, the glass fiber sheets were dried for 30 minutes in an oven at 37° C. under an atmosphere with a controlled humidity of less than 30% in order to dry them.

The cards were assembled by adhesively bonding the conjugate-impregnated glass fiber onto the lower adhesive part of the card while causing the glass fiber sheet to overlap by 1 to 3 mm on the nitrocellulose membrane. In the same way, a paper with a high absorption capacity (absorbent pad zone) (Ahlstrom 222, MAPDS-0100, Arista Biologicals) having dimensions of 30 cm×1.6 cm was placed on the upper part of the card and overlaps the nitrocellulose membrane by 1 to 3 mm in order to be able to create a migration flow.

The card thus assembled was cut into strips 4 mm wide using a guillotine (model CM4000, Biodot).

The strips were then placed in a plastic support called a cassette (MK001, D2 Technologies (registered trademark))

The cassettes were then closed using a clipping device (Closure-I, A-Point Technologies (registered trademark)).

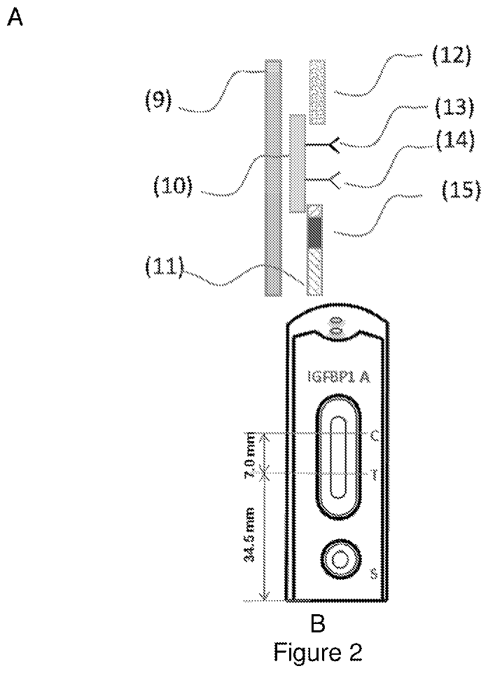

FIG. 2 represents a strip and a support including the strip for the detection of IGFBP-1 A.

### Preparation of the IGFBP-1 B Strip

The strips were prepared from laminated cards of 30 cm×6 cm (CNPC-SS12, MDI (registered trademark)) including or consisting of a plastic support covered with a layer of adhesive on which the nitrocellulose membrane, the absorbent paper and the glass fiber were assembled.

Using an automated reagent dispenser (Isoflow Dispenser, Imagene Technology Inc. (registered trademark)), goat anti-mouse IgG antibodies (ABGAM-0500, Arista Biologicals (registered trademark)) diluted to 0.5 mg/ml in a PBS buffer and also anti-IGFBP-1 antibodies (catalog number 4IG8, clone C7B9, Hytest (registered trademark)) diluted to 0.5 mg/ml in a PBS buffer were deposited, with a flow rate of 1 μl/cm, in the form of parallel lines 5 mm apart and from 1 to 2 mm wide each, on a nitrocellulose membrane, the width of which is 25 mm.

After deposition of these searching methods, these cards were then placed for 30 minutes in an oven at 37° C. under an atmosphere at a controlled humidity of less than 30% in order to dry them.

The conjugate mixture, that is to say a solution including labeled antibodies, was prepared from the anti-IGFBP-1 antibodies (catalog number 4IG8, clone G5F8, Hytest (registered trademark)) and the anti-IGFBP-1 antibodies (catalog number 4I52, clone G2, Hytest (registered trademark)) coupled beforehand to colloidal gold particles according to the following protocol:

1.0 ml of 1% gold chloride solution (G4022, Sigma Aldrich) was added to 100 ml of distilled water. The water was heated to boiling point and 2.5 ml of 1% sodium citrate solution (S1804, Sigma Aldrich) were added. The solution became colorless and then turned purple. After one minute, the solution was heated until it became cherry red. 9.5 ml of 1% sodium citrate (S1804, Sigma Aldrich) were added. The water was heated to boiling point and 8.6 ml of 1% gold chloride solution (G4022, Sigma Aldrich) were added. The solution became dark blue, virtually black, then purple and was then left to cool to ambient temperature.

The pH of 10 ml of the purple colloidal gold solution previously obtained was adjusted, on the one hand, using a 0.2 M potassium carbonate solution (269619, Sigma Aldrich), until a pH of 8.8 was reached; and the pH of 10 ml of purple colloidal gold solution was adjusted to pH 7.8, on the other hand, using a 0.2 M potassium carbonate solution (269619, Sigma Aldrich). 200 μg of anti-IGFBP-1, clone G2, were added to 1 ml of distilled water and then 10 ml of purple colloidal gold solution at the pH adjusted to 8.8 were rapidly added. 200 μg of anti-AIGFBP-1, clone F8, were added to 1 ml of distilled water and then 10 ml of purple colloidal gold solution at the pH adjusted to 7.8 were rapidly added. These tubes were placed on a circular mixer (multidisk rotator 5-50 rpm, Sobinco (registered trademark)) for 20 minutes. 1.5 ml of Gold Stabilization Buffer solution (Artron Bioresearch (registered trademark)) were added. The 2 tubes were centrifuged (5804, Eppendorf (registered trademark)) at 4000 rcf for 30 minutes. The supernatant was suctioned off using a vacuum pump (159600, Brand (registered trademark)) and the pellet was taken up with 1 ml of resuspension buffer (pH 8.0) containing 20 mM Tris Base (26-128-3094, Euromedex (registered trademark)), 50 mM NaCl (S7653, Sigma Aldrich (registered trademark)), 0.2% BSA (1035-70, ID Bio (registered trademark)), 10% sucrose (S8501, Sigma Aldrich), 5% trehalose (T9531, Sigma Aldrich (registered trademark)).

825 μl of the saturating solution ((pH 7.4) 50 mM Na₂HPO₄ (S7907, Sigma Aldrich (registered trademark)), 1% of bovine serum albumin (BSA) (1035-70, ID Bio), 20% of sucrose (S8501, Sigma Aldrich) and 5% of trehalose (T9531, Sigma Aldrich (registered trademark))) were added to 125 μl of solution of IGFBP-1 conjugate, clone F8, to which 50 μl of IGFBP-1 conjugate, clone G2, had been added. The mixture thus obtained was stirred for 20 minutes at ambient temperature on the circular mixer adapted to 1.5 ml tubes.

The mixture of antibodies coupled to the colloidal gold particles was then sprayed, with a flow rate of 4 μl/cm, onto glass fiber sheets (sample conjugate pad zone) having dimensions of 30 cm 3.2 cm (#8964 Ahlstrom, MAPDS-0300, Arista Biologicals (registered trademark)) pretreated with a solution of 0.1 M B₄Na₂O₇ (B3545, Sigma Aldrich (registered trademark)), 1% triton X-100 (X-100, Sigma Aldrich (registered trademark)), pH 8.4.

The binding of this conjugate mixture to the glass fiber sheet was carried out with the same Isoflow Dispenser (registered trademark) apparatus while complying with the manufacturer's indications. After spraying, the glass fiber sheets were dried for 30 minutes in an oven at 37° C. under an atmosphere at a controlled humidity of less than 30% in order to dry them.

The cards were assembled by adhesive bonding of the conjugate-impregnated glass fiber onto the lower adhesive part of the card while making the glass fiber sheet overlap by 1 to 3 mm on the nitrocellulose membrane. In the same way, a paper with a high absorption capacity (absorbent pad zone) (Ahlstrom 222, MAPDS-0100, Arista Biologicals) having dimensions of 30 cm×1.6 cm was placed on the upper part of the card and overlapped the nitrocellulose membrane by 1 to 3 mm in order to be able to create a migration flow.

The card thus assembled was cut into strips 4 mm wide using a guillotine (model CM4000, Biodot).

The strips were then placed in a plastic support called a cassette (MK001, D2 Technologies (registered trademark)).

The cassettes were then closed using a clipping device (Closure-I, A-Point Technologies (registered trademark)).

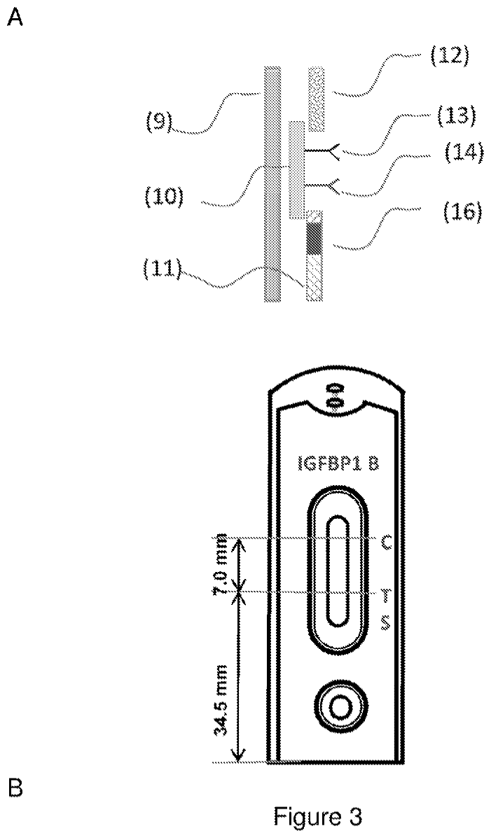

FIG. 3 represents a strip and a support including the strip for the detection of IGFBP-1 A.

### Example 2: Example of Use of a Device for Implementing the Method of Some Embodiments

1. A solution of 1 ml of 50 mM Na₂HPO₄ (S7907, Sigma Aldrich), 1% of bovine serum albumin (BSA) (1035-70, ID Bio), 0.05% triton X-100 (X-100, Sigma Aldrich (registered trademark)). 0.55% of tween 20 (P7949, Sigma Aldrich (registered trademark)), 0.1% of NaN₃ (58032, Sigma Aldrich (registered trademark)) (pH-7.4) was introduced into a dropper bottle (015610, Alltest),
2. the sample was taken with a sterile swab, the bud of which is made of polyester, then deposited in the dropper bottle containing the solution.
3. the mixture obtained was introduced into the wells of the cassettes described in example 1,
4. the result is obtained after 10 minutes of migration.

The test is validated when a colored band appears at the level of the control line.

The test is positive when a colored band appears at the level of at least two test zones (IL-6, IGFBP-1 A and/or IGFBP-1B).

### Example 3: Method for Detecting Imminent Birth

In this example, pregnant women who receive care in the five tertiary hospitals in the south of Nigeria were included. The study was approved by the Conseil Institutionnel Examen [Examining Institutional Council] of NAUTH (letter of approval NAUTH/cs/66/VOL 8/56), and other participating hospitals. Any person who accepted to participate in the study gave an informed consent.

The diagnosis of risk of imminent birth and of preterm birth was carried out in compliance with the protocol of the institutions, including women with intact fetal membranes, with contractions with or without increase in intensity and/or in frequency, a partially or totally effaced cervix, a cervix at less than 3 cm. Pregnant women with a gestational age of 24+0 to 36÷6 presenting symptoms or signs of risk of preterm labor were included. These women presented self declared symptoms or signs, or complaints suggestive of preterm labor.

For admission, the gestational age established on the basis of the ultrasound of the first or second trimester had to be in agreement with the menstrual dates. Women for whom there was a divergence was in particular a final menstrual period and the gestational age at the ultrasound ·10 days to ·20 weeks. The exclusion criteria were composed of multiple pregnancy, polyhydramnios, premature rupture of fetal membranes, triplets, prior cervical examination, sexual intercourse within 24 hours, tocolysis, vaginal bleeding, cervical cerclage, placenta previa, or a known congenital abnormality of the fetus, women suffering from chronic diseases (hypertension, diabetes, renal or cardiac diseases) or genital tract abnormalities were excluded. Pregnancies with delivery less than 14 days post-recruitment for maternal and/or fetal indications were also excluded.

Following the inclusion study, the women underwent a speculum examination carried out while vaginal smear samples were taken in order to measure/detect total IGFBP-1, fetal fibronectin and interleukin 6, prior to the cervical examination. The cervico-vaginal samples were collected during a sterile speculum examination using a Dacron swab by placing the lower end of the swab shaft in the posterior cul de sac of the vagina for 30 s (so as to allow saturation of the pad). After the collection, the pad was inserted into a tube containing 1 ml of extraction buffer (see FIG. 1). The results were interpreted after waiting for 10 minutes. In this example, the three markers fibronectin, IGFBP-1 and interleukin 6 were studied. To do this, anti-fibronectin monoclonal antibodies and/or polyclonal antibodies, and IGFBP-1 and IL-6 antibodies coupled with a blue marker, a pink marker and pink microspheres were used.

The antibodies used were: for IGFBP-1 A, anti-IGFBP-1 antibodies sold by the company Hytest (catalog number 4IG8; clone G5F8 and clone C7B9), for IGFBP-1 B, antibodies sold by the company Hytest (catalog number 4I52; clone G2; catalog number 4IG8; clone G5F8 and clone C7B9), for IL-6, the antibodies sold by Diaclone; catalog number 855.050.005, clone B-E8 or B-E4. The antibodies used for detecting the fibronectin (A and B) were polyclonal antibodies produced in rabbit, sold by the company Sigma under the reference F3648 (Sigma).

For the fibronectin and the IGFBP-1, two different forms of these proteins were used as markers: fibronectin A and fibronectin B, and IGFBP-1 A and IGFBP-1 B. After measurement, a monitoring of birth or of the possibility of birth was performed 2, 7 and 14 days later. A comparison of the results via the detection of the markers: IGFBP-1 A, IGFBP-1 B and IL-6 with the detection of fibronectin A and B was carried out. The test was considered to be positive if at least two of the three biomarkers IGFBP-1, IGFBP-1 B and IL-6 were present in the sample.

The cervical measurements were carried out by transvaginal ultrasound. During a gynecological examination, the speculum examination was carried out by introducing a Cusco speculum into the vagina before touching the vagina. The parameters recorded during the pelvic examination included cervical dilation, membrane state, cervical effacement, patient history, namely age, number of births and miscarriages, and the results of the triplex tests.

The cervical examinations preceded each ultrasound examination. To minimize the intra-observer variability of the cervical measurement, the women had to empty their bladder just before the ultrasound and the sagittal cervical canal was perfectly identified so that the internal orifice and the external orifice were both identified and the actual length corresponding to the distance corresponding to the straight line between them was measured.

### Statistical Analyses

The size of the sample was calculated as being 94 with a power of 80%, ·=0.05, ·=20%, and a standard value of the effect of 0.84. The Student's t test was used to analyze the continuous variables. The statistical significance of the differences between the groups for the continuous variables was evaluated and the differences in proportions were evaluated using the Mantel-Haenszel test. The data were analyzed with Epi Info Version 3.5.1 (Centers for Disease Control and Prevention, Atlanta, Ga., USA) and Stata version 10 (StataCorp, College Station, Tex., USA). The values of P<0.05 were considered to be statistically significant.

The sensitivity and specificity, the positive and negative predictive values and the accuracy values were also determined. The sensitivity was calculated as follows: true positive (TP)/[TP+false negatives (FN)]; and the specificity was calculated as follows: true negative (TN)/[TN+false positives (FP)]. The positive predictive value (PPV)=TP/(TP+FP), while the negative predictive value NPV=TN/(TN+FN). In addition, the accuracy=([true positive+true negative]/[true negative+false negative+false positive+true negative]+100%) of each test was also estimated. The probability of a preterm birth was measured by calculating the relative risk (RR) ratio and the respective confidence intervals (CI) at 95% for each test.

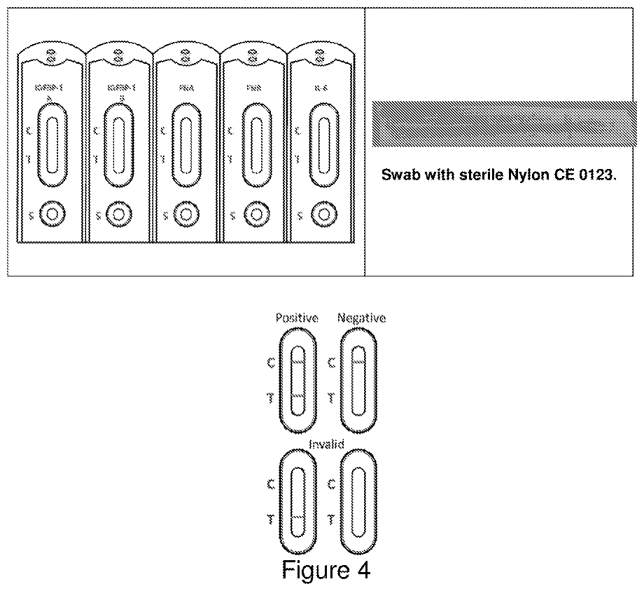

117 women with single pregnancies were evaluated for admissibility to participate in the study and 98 of them were recruited. Of the 98 women who were recruited, three women were excluded (two women had non-valid results and one woman underwent a planned Cesarean section owing to an iatrogenic factor before the 14 days of the monitoring period and their data were therefore excluded from the statistical analysis). A total of 95 women were finally included in the analysis. The model for inclusion of women participating in the study is shown in FIG. 4.

For the women included, the average age was 31.1±2.3 years old (extremes: 20-40). The mean gravidity and the mean parity were respectively 2.5±0.9 (range of 1-6) and 1.4±0.4 (range of 0-5). The mean gestational age at inclusion was 34.1±1.8 weeks (range of 24-36 weeks).

The matrices of performance of the tests (IGFBP-1 A, IGFBP-1 B and IL-6 with respect to fibronectin) in the 2 days, 7 days and 14 days of inclusion in the study are presented respectively in table 1, table 2 and table 3. Overall, the predictive accuracy of the combined tests with respect to the days of inclusion was: 85.3% (·2 days), 93.7% (·7 days) and 94.7% (·14 days) which is higher than the accuracy with fibronectin A. (71.6%. 82.1% and 85.3%) 75 and of fibronectin B (57.9%, 70.5% and 71.6%) respectively.

Of the 95 patients included in the analysis, 25 (26.3%) gave birth before 37 weeks of gestation and 70 (73.7%) gave birth at 37 weeks of gestation or later. The matrix of performance of IGFBP-1 A/IGFBP-1 B and IL-6 with respect to fibronectin as a function of the delivery time is described in table 4.

The mean body temperature for the women included in the study was 36.8±0.4° C. The mean cervical length by ultrasound for the women who gave birth in the 14 days following inclusion in the study was 24.4±1.3 mm compared with 32.5±1.9 mm in the women who did not give birth in the 14 days following inscription (T=−34.29; p<0.001).

TABLE 1
Results of the combination IGFBP-1 A/IGFBP-1 B and IL-6 with respect
to fibronectin (A/B) of the tests in women in preterm labor with a
risk of giving birth at * 2 days after inclusion in the study

| | | | | | | |
|---|---|---|---|---|---|---|
| | 2/3 among | Fibonectin | Fibonectin | | | |
| | IGFRP-1 | A | B | | | |
| | A/ | (detection | (detection | | | |
| | IGFBP-1 | threshold | threshold | IGEBP- | IGFBP- | |
| Markers | B & IL-6 | 500 ng/ml) | 50 ng/ml) | I A | 1 B | IL-6 |

Criteria

| | | | | | | |
|---|---|---|---|---|---|---|
| True | 62 | 51 | 38 | 68 | 61 | 52 |
| negative | | | | | | |
| True | 19 | 17 | 17 | 16 | 18 | 19 |
| positive | | | | | | |
| False | 0 | 3 | 2 | 4 | 2 | 1 |
| negative | 14 | 25 | 38 | 9 | 14 | 24 |
| False | | | | | | |
| positive | | | | | | |
| Total | 95 | 95 | 95 | 95 | 95 | 95 |
| % | 81.6 | 67.1 | 50 | 88.3 | 81.3 | 68.4 |
| speci- | | | | | | |
| ficity | | | | | | |
| Sensi- | 100 | 85 | 89.5 | 80 | 90 | 95 |
| tivity | | | | | | |
| Negative | 100 | 94.4 | 95 | 94.4 | 96.8 | 98.1 |
| predictive | | | | | | |
| value | | | | | | |
| Positive | 57.6 | 40.5 | 30.9 | 64 | 56.3 | 44.2 |
| predictive | | | | | | |
| value | | | | | | |
| Accuracy | 85.3 | 71.6 | 57.9 | 88.4 | 83.2 | 74.7 |

The accuracy of 85.3% was statistically significant with respect to the accuracy of fibronectin A of 71.6% (Mantel-Haenszel chi squared=5.23, p=0.02, relative risk=1.59, CI at 95%=01.02 to 02.25) or of fibronectin B of 57.9% (Mantel-Haenszel chi squared=17.40, p<0.001, risk ratio=2.30, CI at 95%=1.43 to 3.68).

TABLE 2
Results of the combination IGFBP-1 A/IGFBP-1 B and IL-6 with
respect to fibronectin (A/B) of the tests in the women in preterm
labor with a risk of giving birth at * 7 days after inclusion in the study

| | | | | | | |
|---|---|---|---|---|---|---|
| | 2/3 among | Fibronectin | Fibronectin | | | |
| | IGFBP-1 | A | B | | | |
| | A/ | (detection | (detection | | | |
| | IGFBP-1 | threshold | threshold | IGFBP- | IGFBP- | |
| Markers | B & IL-6 | 500 ng/ml) | 50 ng/ml) | 1A | 1B | IL-6 |

Criteria

| | | | | | | |
|---|---|---|---|---|---|---|
| True | 60 | 50 | 38 | 63 | 60 | 50 |
| negative | | | | | | |
| True | 29 | 28 | 29 | 23 | 28 | 29 |
| positive | | | | | | |
| False | 4 | 3 | 2 | 8 | 2 | 2 |
| negative | | | | | | |
| False | 4 | 14 | 26 | 1 | 5 | 14 |
| positive | | | | | | |
| Total | 95 | 95 | 95 | 95 | 95 | 95 |
| % | 93.8 | 78.1 | 59.4 | 98.4 | 96.8 | 78.1 |
| speci- | | | | | | |
| ficity | | | | | | |
| Sensi- | 87.9 | 90.3 | 93.5 | 74.2 | 93.3 | 96.2 |
| tivity | | | | | | |
| Negative | 93.8 | 94.3 | 95 | 88.7 | 96.8 | 96.2 |
| predictive | | | | | | |
| value | | | | | | |
| Positive | 87.9 | 66.7 | 52.7 | 95.8 | 84.8 | 67.4 |
| predictive | | | | | | |
| value | | | | | | |
| Accuracy | 93.7 | 82.1 | 70.5 | 90.5 | 92.6 | 83.2 |

The accuracy of 93.7% was statistically significant with respect to the accuracy of fibronectin A of 82.1% (Mantel-Haenszel chi squared=5.95, p=0.01, relative risk=2.04, CI at 95%=1.01 to 4.12) or of fibronectin B of 70.5% (Mantel-Haenszel chi squared=17.25, p<0.001, the risk ratio=3.23, CI at 95%=1.54 to 6.77).

TABLE 3
Results of the combination IGFBP-1 A/IGFBP-1 B and IL-6 with
respect to fibronectin (A/B) of the tests in the women in preterm labor
with a risk of giving birth at * 14 days after inclusion in the study

| | | | | | | |
|---|---|---|---|---|---|---|
| | 2/3 among | Fibronectin | Fibronectin | | | |
| | IGFBP-1 | A | B | | | |
| | A/ | (detection | (detection | | | |
| | IGFBP-1 | threshold | threshold | IGFBP- | IGFBP- | |
| Markers | B & IL-6 | 500 ng/ml) | 50 ng/ml) | 1 A | 1 B | IL-6 |

Criteria

| | | | | | | |
|---|---|---|---|---|---|---|
| True | 59 | 50 | 37 | 61 | 59 | 49 |
| negative | | | | | | |
| True | 31 | 31 | 31 | 23 | 31 | 31 |
| positive | | | | | | |
| False | 3 | 3 | 3 | 10 | 3 | 3 |
| negative | | | | | | |
| False | 2 | 11 | 21 | 1 | 2 | 12 |
| positive | | | | | | |
| Total | 95 | 95 | 95 | 95 | 95 | 95 |
| % | 96.7 | 82 | 60.7 | 98.4 | 96.7 | 80.3 |
| speci- | | | | | | |
| ficity | | | | | | |
| Sensi- | 94.2 | 91.2 | 91.2 | 69.7 | 91.2 | 94.2 |
| tivity | | | | | | |
| Negative | 93.9 | 73.8 | 56.4 | 95.8 | 93.9 | 72.1 |
| predictive | | | | | | |
| value | | | | | | |
| Positive | 93.9 | 73.8 | 56.4 | 95.8 | 93.9 | 72.1 |
| predictive | | | | | | |
| value | | | | | | |
| Accuracy | 94.7 | 85.3 | 71.6 | 88.4 | 94.7 | 84.2 |

The accuracy of 94.7% was statistically significant with respect to the accuracy of fibronectin A of 85.3% (Mantel-Haenszel chi squared=4.71, p=0.03, relative risk=2.00, CI at 95%=0.93 to 4.30) or of test fibronectin B of 71.6% (Mantel-Haenszel chi squared=18.09, p<0.001, risk ratio=3.65, CI at 95%=1.61 to 8.25).

TABLE 4
Result of the combination with respect to the fibronectin test on the cases
of preterm labor as a function of gestational age for the women having
given birth at * 14 days after inclusion in the study

| | | | | | | |
|---|---|---|---|---|---|---|
| | 2/3 among | Fibronectin | Fibronectin | | | |
| | IGFBP-1 | A | B | | | |
| | A/ | (detection | (detection | | | |
| | IGFBP-1 | threshold | threshold | IGFBP- | IGFBP- | |
| Markers | B & IL-6 | 500 ng/ml) | 50 ng/ml) | 1 A | 1 B | 1L-6 |

Criteria

| | | | | | | |
|---|---|---|---|---|---|---|
| True | 59 | 50 | 37 | 61 | 59 | 49 |
| negative | | | | | | |
| True | 31 | 31 | 31 | 23 | 31 | 31 |
| positive | | | | | | |
| False | 3 | 3 | 3 | 10 | 3 | 3 |
| negative | | | | | | |
| False | 2 | 11 | 21 | 1 | 2 | 12 |
| positive | | | | | | |
| Total | 95 | 95 | 95 | 95 | 95 | 95 |
| % | 96.7 | 82 | 60.7 | 98.4 | 96.7 | 80.3 |
| speci- | | | | | | |
| ficity | | | | | | |
| Sensi- | 91.2 | 91.2 | 91.2 | 69.7 | 91.2 | 91.2 |
| tivity | | | | | | |
| Negative | 95.2 | 94.3 | 92.5 | 85.9 | 95.2 | 94.2 |
| predictive | | | | | | |
| value | | | | | | |
| Positive | 93.9 | 73.8 | 56.4 | 95.8 | 93.9 | 72.1 |
| predictive | | | | | | |
| value | | | | | | |
| Accuracy | 94.7 | 85.3 | 71.6 | 88.4 | 94.7 | 84.2 |

Of the 95 patients included in the analysis. 25 (26.3%) gave birth before 37 weeks of gestation and 70 (73.7%) gave birth at or after 37 weeks of gestation. The matrix of performance of IGFBP-1 A/IGFBP-1B and IL-6 with respect to fibronectin as a function of the delivery date is shown in table 2.

The mean body temperature for the women was 36.8±0.4° C. The mean cervical length by ultrasound for the women who gave birth in the 14 days following inclusion was 24.4±1.3 mm compared with 32.5±1.9 mm in the women who did not give birth in the 14 days following inscription (T=−34.29; p<0.001).

As demonstrated in this example, the method of some embodiments in which at least two of the markers are positive makes it possible to obtain results which are more sensitive and more specific with a better positive and negative predictive value than the known reference test using fibronectin [13]. In particular, the method according to some embodiments makes it possible to obtain results with a positive predictive value which is much higher than that using fibronectin.

In particular, as demonstrated in this example, the method of some embodiments makes it possible to obtain a strong sensitivity of 91.2%, with an accuracy and the specificity, positive predictive value and negative predictive value of the combined, test which are higher, for example 25% higher, than for fibronectin A or fibronectin B respectively. The differences in results were statistically significant (p<0.05).

In addition, as demonstrated in this example, the method makes it possible to obtain a sensitivity equal to 100% and a negative predictive value of 100% for a threat of giving birth at two days.

As demonstrated in this example, the method according to some embodiments makes it possible, by virtue of its sensitivity, specificity, and negative and positive predictive value, to accurately identify the women who will experience spontaneous preterm labor.

Specifically, the sensitivity is 91.2% and the specificity is 96.7%. The high negative predictive value, 93.9%, combined with the strong sensitivity and specificity, makes it possible to avoid needless medical obstetric procedures during the pregnancy.

In addition, in the case of a full-term pregnancy, the sensitivity and the specificity were respectively 81.8% and 96.6%, clearly demonstrating that the method according to some embodiments is a method that is of use for predicting a full-term pregnancy.

In addition, an analysis of the results when the three markers are positive and/or negative as a function of the date of the birth was carried out and is presented in table 5 below.

TABLE 5
Result of the test for the women who had or had not given birth
at less than 14, 7 and 2 days after inclusion in the study

| | | Birth < 14 days | | |
|---|---|---|---|---|
| | | No | Yes | Total |
| IGFBP-1 A + | Triple negative | 48 | 1 | 49 |
| IGFBP-1 B + | Triple positive | 1 | 23 | 24 |
| IL-6 | Other result | 17 | 10 | 22 |
| | Total | 61 | 34 | 95 |
| | | Birth < 7 days | | |
| | | No | Yes | Total |
| IGFBP-1 A + | Triple negative | 48 | 1 | 49 |
| IGFBP-1 B + | Triple positive | 1 | 23 | 24 |
| IL-6 | Other result | 16 | 6 | 22 |
| | Total | 65 | 30 | 95 |
| | | Birth < 2 days | | |
| | | No | Yes | Total |
| IGFBP-1 A + | Triple negative | 49 | 0 | 49 |
| IGFBP-1 B + | Triple positive | 14 | 10 | 24 |
| IL-6 | Other result | 19 | 3 | 22 |
| | Total | 82 | 13 | 95 |
| Results with a triple positive | | Birth | | |
| or triple negative result | | < 14 days | < 7 days | < 2 days |
| IGFBP-1 A + | Sensitivity | 95.8% | 95.8% | 100.0% |
| IGFBP-1 B + | Specificity | 98.0% | 98.0% | 77.8% |
| IL-6 | NPV | 98.0% | 98.0% | 100.0% |
| | PPV | 95.8% | 95.8% | 41.7% |
| | Other result | 23.2% | 23.2% | 23.2% |

As demonstrated below, the method of some embodiments advantageously makes it possible to obtain a result with a sensitivity and a specificity equal respectively to 95.8% and 98.0% for a threat of giving birth at 7 or 14 days.

In addition, when the result is triple negative, the negative predictive value for a birth at 7 or 14 days is equal to 98%, and 100% for a birth at 2 days.

Thus, this example clearly demonstrates that the determination of the presence or absence in the vaginal secretions of at least two markers chosen from IGFBP-1 (A and B) and IL-6 is a reliable method for predicting imminent birth in women in preterm labor and has a sensitivity and a specificity for the prediction of imminent birth much higher than the reference methods used clinically. In addition, this example clearly demonstrates that the use of IGFBP-1 and of IL-6 makes it possible to maximize the sensitivity and to minimize the false-positive diagnoses. Furthermore, the method according to some embodiments makes it possible to obtain a “tare” negative with a negative predictive value equal to 98% or 100%, thus making it possible to avoid needless clinical procedures and or pharmacological treatments.

### Example 4: Method for Detecting Imminent Birth

In this example, the protocols for admission, for carrying out the test and for examinations after inclusion and the women included were identical to those of example 3 above.

The search for IGFBP-1 A, IGFBP-1 B and IL-6 was carried out as described in example 3 above.

When IGFBP-1 A was detected, a value I_{A} equal to 2 was assigned; when IGFBP-1 B was detected, a value I_{B} equal to 2 was assigned and when IL-6 was detected, a value I₆ equal to 1 was assigned. In the absence of detection, the value assigned was 0 for each of the markers.

After assignment of the I_{A}, I_{B} and I₆ values, the values were added together according to the score S₁ with the following formula:

S₁=I_{A}+I_{B}+I₆

The values of the score S₁ obtained for the women who had or had not given birth at less than 14, 7 and 2 days after inclusion in the study are summarized in table 6 below.

TABLE 6
Result of the test for the women who had or had not given
birth at less than 14, 7 and 2 days after inclusion in the study

| | | Number of patients having given birth | | |
|---|---|---|---|---|
| Total | Number of | Under | Under | Under |
| score S₁ | patients | 2 days | 3-7 days | 8-14 days |
| 0 | 50 | 0 | 1 | 0 |
| 1 | 12 | 1 | 0 | 0 |
| 1-4 | 11 | 2 | 4 | 4 |
| 5 | 24 | 16 | 7 | 0 |

On the basis of the results described in table 6 above, the calculated probabilities of giving birth were calculated from the results obtained as a function of the score obtained and are summarized in table 7 below.

TABLE 7
Probability of giving birth as a function of the score

| | Number | Probability of giving birth | | | | | |
|---|---|---|---|---|---|---|---|
| Score | of | Under 2 | | Under 7 | | Under 14 | |
| S₁ | patients | days | | days | | days | |
| 0 | 50 | 0% | | 2% | (1/50) | 2% | (1/50) |
| 1 | 12 | 8.30% | (1/2) | 8.30% | (1/12) | 8.30% | (1/12) |
| 1-4 | 11 | 18.20% | (2/11) | 54.50% | (6/11) | 90.90% | (10/11) |
| 5 | 24 | 66.70% | (16/24) | 95.80% | (23/24) | 95.80% | (23/24) |

As illustrated in table 7 above, a score S1 greater than or equal to 2 implies a probability of giving birth within the next 14 days of greater than 90.90%.

The negative and positive predictive values were calculated according to the following formulae:

PPV=TP/(TP+FP) and NPV=TN/(TN+FN)

in which TP signifies true positive, FP signifies false positive. TN signifies true negative and FN signifies false negative.

In addition, an evaluation of the negative and positive predictive value when the score S1 obtained was greater than or equal to 2 as a function of the time until birth, less than or equal to 2 days, less then or equal to 7 days or less than or equal to 14 days, was carried out. Table 8 below summarizes the results obtained.

TABLE 8
Negative or positive predictive values with a score greater
than or equal to 2

| | Time until birth | | |
|---|---|---|---|
| | Less than | Less than | Less than |
| | or equal to | or equal to | or equal to |
| Score S₁ · 2 | 2 days | 7 days | 14 days |
| Positive | 51.4% | 82.9% | 94.3% |
| predictive value | | | |
| (PPV) | | | |
| Negative | 98.4% | 96.8% | 96.8% |
| predictive value | | | |
| (NPV) | | | |

As demonstrated in table 8 above, regardless of the imminent nature of the birth, for example less than 2 days, less than 7 days and/or less than 14 days, when the score S₁ is greater than or equal to 2, the negative predictive value is greater than 96.8% ranging to 98.4%.

An evaluation of the negative predictive value when the score S1 obtained was equal to 0 or of the positive predictive value when the score S1 obtained was equal to 5 as a function of the time until birth, less than or equal to 2 days, less than or equal to 7 days or less than or equal to 14 days, was carried out. Table 9 below summarizes the results obtained.

TABLE 9
Negative predictive value with a score equal to 0 and positive
predictive value with a score equal to 5

| | | Time until birth | | |
|---|---|---|---|---|
| Score S₁ | | · 2 days | · 7 days | · 14 days |
| 5 | PPV | 66.7% | 95.8% | 95.8% |
| 0 | NPV | 100.0% | 98.0% | 98.0% |

In addition, as demonstrated in table 9 above, the method makes it possible to obtain, a negative predictive value of 100% for a threat of giving birth at two days and equal to 98% for giving birth in the next 14 days.

The example is also carried out with a different assignment of values for each of the markers IGFBP-1 A, IGFBP-1 B and IL-6. Table 10 below summarizes the various assignments, distinguishing values of the score and associated results.

TABLE 10
Assignment of the values and calculation of the corresponding scores

| | | | | | Time until birth | | |
|---|---|---|---|---|---|---|---|
| | | | | | Less than or | Less than or | Less than or |
| Markers | Present | Absent | S₁ = I_{A} + I_{B} + I₆ | | equal to 2 days | equal to 7 days | equal to 14 days |
| IGFBP-1 A | 2 | 0 | S1 = 0 | NPV | 100.0% | 98.0% | 98.0% |
| I_{A} | | | | | | | |
| IGFBP-1 B | 2 | 0 | S₁ · 2 | PPV | 51.4% | 82.9% | 94.3% |
| I_{B} | | | S₁ < 2 | NPV | 98.4% | 96.8% | 96.8% |
| IL-6 | 1 | 0 | S1 = 5 | PPV | 66.7% | 95.8% | 95.8% |
| I₆ | | | | | | | |
| Markers | Present | Absent | S₁ = I_{A} + I_{B} + I₆ | | 2 days | 7 days | 14 days |
| IGFBP-1 A | 3 | 0 | S1 = 0 | NPV | 100.0% | 98.0% | 98.0% |
| I_{A} | | | | | | | |
| IGFBP-1 B | 2 | 0 | S₁ · 2 | PPV | 51.4% | 82.9% | 94.3% |
| I_{B} | | | S₁ < 2 | NPV | 98.4% | 96.8% | 96.8% |
| IL-6 | 1 | 0 | S1 = 6 | PPV | 66.7% | 95.8% | 95.8% |
| I₆ | | | | | | | |
| Markers | Present | Absent | S₁ = I_{A} + I_{B} + I₆ | | 2 days | 7 days | 14 days |
| IGFBP-1 A | 3 | 0 | S1 = 0 | NPV | 100.0% | 98.0% | 98.0% |
| I_{A} | | | | | | | |
| IGFBP-1 B | 3 | 0 | S₁ · 3 | PPV | 51.4% | 82.9% | 94.3% |
| I_{B} | | | S₁ < 3 | NPV | 98.4% | 96.8% | 96.8% |
| IL-6 | 1 | 0 | S1 = 7 | PPV | 66.7% | 95.8% | 95.8% |
| I₆ | | | | | | | |
| Markers | Present | Absent | S₁ = I_{A} + I_{B} + I₆ | | 2 days | 7 days | 14 days |
| IGFBP-1 A | 4 | 0 | S1 = 0 | NPV | 100.0% | 98.0% | 98.0% |
| I_{A} | | | | | | | |
| IGFBP-1 B | 3 | 0 | S₁ · 3 | PPV | 51.4% | 82.9% | 94.3% |
| I_{B} | | | S₁ < 3 | NPV | 98.4% | 96.8% | 96.8% |
| IL-6 | 1 | 0 | S1 = 8 | PPV | 66.7% | 95.8% | 95.8% |
| I₆ | | | | | | | |

As demonstrated in his example, the method according to some embodiments makes it possible, by virtue of its sensitivity, specificity, negative predictive value and positive predictive value, to accurately identify the women who will give birth within a predefined period of time.

In addition, from the very high negative predictive value, namely greater than 98% or even equal to 100%, the method according to some embodiments makes it possible to reduce the false-negative results and therefore provides a reliable result which can be used by clinicians.

As demonstrated in this example, the method according to some embodiments makes it possible, by virtue of its sensitivity, specificity, negative predictive value and positive predictive value, to accurately identify the women who will give birth within a predefined period of time, making it possible to avoid needless hospitalizations and medical obstetric procedures during the pregnancy.

Thus, this example clearly demonstrates that the method including the calculation of the score S₃ according to some embodiments is a reliable method for predicting imminent birth in women in preterm labor and has a sensitivity and a specificity for predicting imminent birth which are much higher than the reference methods used clinically.

Furthermore, this example clearly demonstrates that a method according to some embodiments makes it possible to obtain a “true” negative with a negative predictive value equal to 98% or 100%, thus making it possible to avoid needless hospitalizations, clinical procedures and/or pharmacological treatments.

### LIST OF REFERENCES

1. Eroglu D, Yanik F, Oktem M, Zeyneloglu H B, Kuscu E. Prediction of preterm delivery among women with threatened preterm labor. 2007; 64(2):109-16. Epub 2007 Feb. 23.
2. Berghella V. Novel developments on cervical length screening and progesterone for preventing preterm birth. BJOG. 2009 January; 116(2):182-7. doi: 10.1111/j.1471-0528.2008.02008.x.
3. Berghella V, Baxter J K, Hendrix N W. Cervical assessment by ultrasound for preventing preterm delivery. Cochrane Database Syst Rev. Jan. 31, 2013; 1:CD007235. doi: 10.1002/14651858.CD007235.pub3.
4. Goepfert A R, Goldenberg R L, Andrews W W, Hauth J C, Mercer B, et al. The Preterm Prediction Study: association between cervical interleukin 6 concentration and spontaneous preterm birth. National Institute of Child Health and Human Development Maternal-Fetal Medicine Units Network. Am J Obstet Gynecol. 2001 February; 184(3):483-8.
5. Leitich H, Egarter C, Kaider A, Hohlagschwandtner M, Berghammer P, Husslein P. Cervicovaginal fetal fibronectin as a marker for preterm delivery: a meta-analysis. Am J Obstet Gynecol. 1999 May; 180(5):1169-76.
6. Goldenberg R L, Mercer B M, Meis P J, Copper R L, Das A, McNellis D. The preterm prediction study: fetal fibronectin testing and spontaneous preterm birth. NICHD Maternal Fetal Medicine Units Network. Obstet Gynecol. 1996 May; 87(5 Pt 1):643-8,
7. McLaren J S, Hezelgrave N L, Ayubi H, Seed P T, Shennan A H. Prediction of spontaneous preterm birth using quantitative fetal fibronectin after recent sexual intercourse. Am J Obstet Gynecol, 2015 January; 212(1):89.e1-5. Epub 2014 Jun. 30.
8. Kurkinen-Räty M, Ruokonen A, Vuopala S, Koskela M, Rutanen E M, Kärkkäinen T, Jouppila P. Combination of cervical interleukin-6 and -8, phosphorylated insulin-like growth factor-binding protein-1 and transvaginal cervical ultrasonography in assessment of the risk of preterm birth. BJOG. 2001 August; 108(8):875-81.
9. Rutanen et al, Biochem Biophys Res Commun 1988; 152:208
10. Pekonen et al. J immunoassay 1989; 10:325-337
11. Beer et al. Qualification of cellulose nitrate membranes for lateral-flow assays, IVD technology, January 2002
12. Anne Harwood Peruski et al. Immunological Methods for Detection and Identification of Infectious Disease and Biological Warfare Agents. Clinical and diagnostic laboratory immunology, July 2003, p. 506-513
13. Honest H, Forbes C A, Duree K H, et al. Screening to prevent spontaneous preterm birth: systematic reviews of accuracy and effectiveness literature with economic modelling. Health Technol Assess 2009; 13:1-627.

1. A method for the in vitro detection/prediction of an imminent birth, comprising:
simultaneously searching, in a specimen of vaginal or cervical secretions, for markers of a the group consisting of intact insulin-like growth factor binding protein 1 (IGFBP-1 A), of intact insulin-like growth factor binding protein 1 and/or an N-terminal fragment of insulin-like growth factor binding protein 1 (IGFBP-1 B) and of interleukin 6 (IL-6); and
detecting or predicting an imminent birth if at least two of the markers of the group consisting of insulin-like growth factor binding protein 1 A (IGFBP-1 A), of insulin-like growth factor binding protein 1 B (IGFBP-1 B) and of interleukin 6 (IL-6), are detected.

2. A method for the in vitro detection/prediction of an imminent birth in a sample of vaginal or cervical secretions, comprising:
a) searching for the presence of IGFBP-1 A, of IGFBP-1 B and of IL-6,
b) assigning a value of IGFBP-1 A (I_{A}) if IGFBP-1 A is present in the sample or of 0 if IGFBP-1 A is not present,
c) assigning a value of IGFBP-1 B (I_{B}) if IGFBP-1 B is present in the sample or of 0 if IGFBP-1 B is not present, the value of I_{B} assigned if IGFBP-1 B is present in the sample being less than or equal to the value of I_{A} assigned when IGFBP-1 A is present in the sample,
d) assigning a value of IL-6 (I₆) if IL-6 is present in the sample or of 0 if IL-6 is not present, the value I₆ assigned when IL-6 is present in the sample being strictly less than the values of I_{A} and I_{B} assigned when IGFBP-1 A and IGFBP-1 B are respectively present in the sample,
e) calculating the score S1 according to the following formula:
S₁=I_{A}+I_{B}+I₆
a value of S1 greater than or equal to the value of I_{A} or I_{B}, respectively assigned when IGFBP-1 A or IGFBP-1 B is present in the sample, indicating an imminent birth.

3. The method as claimed in claim 1, wherein the searching is carried out with at least one capture antibody directed against IL-6, at least one capture antibody directed against IGFBP-1 A and at least one capture antibody directed against IGFBP-1 B.

4. The method as claimed in claim 2, wherein the searching is carried out using a sample of vaginal or cervical secretions diluted in a buffer solution.

5. The method as claimed in claim 1, wherein the searching is carried out using a specimen of vaginal or cervical secretions diluted in a buffer solution.

6. The method as claimed in in claim 2, further comprising at least one of the parameters chosen from the volume, the pH and the ionic strength of the buffer medium.

7. The method as claimed in claim 5, further comprising adjusting at least one of the parameters chosen from the concentration of the capture antibodies, the adjustment being specific to IL-6, to IGFBP-1 A or to IGFBP-1 B.

8. The method as claimed in claim 5, further comprising adjusting at least one of the parameters chosen from the concentration of the capture antibodies, the adjustment being specific to IL-6, to IGFBP-1 A and/or to IGFBP-1 B.

9. The method as claimed in claim 5, wherein the pH of the buffer solution is from 5 to 10.

10. The method as claimed in claim 1, wherein the searching step includes a revealing step carried out with at least one labeled anti-IGFBP-1 A antibody, at least one labeled anti-IGFBP-1 B antibody and at least one labeled anti-IL-6 antibody.

11. The method as claimed in claim 1, wherein the IGFBP-1 A is detected starting from a concentration of 40 ng/ml.

12. The method as claimed in claim 1, wherein the IGFBP-1 B is detected starting from a concentration of 20 ng/ml intact and/or 10 ng/ml of the N-Ter fragment of IGFBP-1.

13. The method as claimed in claim 1, wherein the IL-6 is detected starting from a concentration of 0.25 ng/ml.

14. A device for implementing the method as claimed in claim 1, comprising:
a sample-depositing zone 1,
a zone 2 including labeled anti-IGFBP-1 A antibodies, labeled anti-IGFBP-1 B antibodies and labeled anti-IL-6 antibodies,
a revealing zone 3 including capture antibodies directed against IGFBP-1 A,
a revealing zone 4 including capture antibodies directed against IGFPB-1 B,
a revealing zone 5 including capture antibodies directed against IL-6.

15. The device as claimed in claim 14, wherein the depositing zone 1 is chosen from the group including an absorbent paper and a glass fiber support.

16. The device as claimed in claim 14, wherein the revealing zone 4 and/or 5 is a nitrocellulose membrane.

17. The device as claimed in claim 14, wherein the zone 2 and/or 3 is a glass fiber support.

18. The device as claimed in claim 14, wherein the zones 1, 2, and/or 3 are a glass-fiber or nitrocellulose support.

19. (canceled)

20. The method as claimed in claim 2, wherein the searching is carried out with at least one capture antibody directed against IL-6, at least one capture antibody directed against IGFBP-1 A and at least one capture antibody against IGFBP-1 B.

21. The method as claimed in claim 3, wherein the searching is carried out using a sample of vaginal or cervical secretions diluted in a buffer solution.
